(19) United States
(12) Patent Application Publication
(10) Pub. No.: US 2007/0166255 A1
(43) Pub. Date: Jul. 19, 2007

(54) Treatment of Topical Discomforts Including Acne, Sunburn, Diaper Rash, Wound, Wrinkles and Dandruff/Hair Loss by Natural Lignans via Fatty Acid Desaturase Inhibition

(76) Inventor: SHYAM K GUPTA, Scottsdale, AZ (US)

Correspondence Address: SHYAM K. GUPTA;BIODERM RESEARCH, 5221 E. WINDROSE DRIVE, SCOTTSDALE, AZ, 85254, US

(21) Appl. No.: 11/670,942

(22) Filed: Feb. 2, 2007

Related U.S. Application Data
(63) Continuation-in-part of application No. 11/161,856, filed on Aug. 19, 2005.
(63) Continuation-in-part of application No. 10/904,665, filed on Nov. 22, 2004.

(51) Int. Cl. A61K 8/49; A61K 8/31

(52) U.S. Cl. 424/70.1; 514/473; 514/731; 514/754

(57) ABSTRACT

The present invention discloses compositions and methodology based on certain natural lignans that provide treatment of topical discomforts and ailments including sunburn, radiation burn, thermal burn and blisters, diaper rash, shaving burn, wound, venous cannulation, acne, darkened skin pigmentation, skin wrinkles, dandruff, and hair loss; said treatment having achieved via topical inhibition of Fatty Acid Desaturases.

This is a continuation-in-part of U.S. patent application Ser. No. 11/161,856; filed Aug. 19, 2005; the said application having disclosed certain furostanol saponins and sapogenins that reduce sebum production. This is also a continuation-in-part of U.S. patent application Ser. No. 10/904,665; filed Nov. 22, 2004; the said application having disclosed a topical delivery system for cosmetic and pharmaceutical agents.

The present invention discloses compositions and methodology for topical application based on certain natural lignans that provide treatment of topical discomforts and ailments including sunburn, radiation burn, thermal burn and blisters, diaper rash, shaving burn, wound, venous cannulation, acne, darkened skin pigmentation, skin wrinkles, dandruff, and hair loss, said treatment achieved via topical inhibition of Fatty Acid Desaturases.

Topical discomfort relates to certain conditions that can cause either physical pain and anxiety or mental (psychological) anguish. In most cases these are not life-threatening situations. The examples of such physical pain and anxiety include sunburn, radiation burn, thermal burn and blisters, shaving burn, wound, venous cannulation, and acne. The examples mental or psychological anguish include dandruff, hair loss, skin wrinkles, and darkened skin pigmentation.

Fatty Acid Desaturases are enzymes that catalyze the insertion of a double bond at the Delta position of fatty acids. There seem to be two different families of fatty acid desaturases. The fatty acid desaturases of interest in the present invention are those belonging to family-2 of the desaturases, some of which are also called Delta-5-desaturases and Delta-6-desaturases, or fatty acid desaturase-2. Fatty acid desaturases in skin have been unknown, until very recently. The desaturases thus appear to work in a direction opposite to that of dehydrogenases, for example, 5-Alpha-reductase, which causes the hydrogenation of the double bond in testosterone to form dihydrotestosterone (DHT), which is biologically more active than testosterone.

Delta6-desaturase reaction, for example, is the rate-limiting step in the conversion of linoleic acid and alpha linoleic acids to the longer, more highly unsaturated members of the n-6 and n-3 polyunsaturated fatty acids in the metabolic pathway of mammalian cells. Delta6-desaturase is also required for the conversion of dietary linoleic acid to arachidonic acid. Delta6-desaturase has recently been shown to catalyze an unexpected “sebaceous-type” reaction, that of converting palmitate into the mono-unsaturated fatty acid, sapienate, a 16-carbon fatty acid with a single cis double bond at the sixth carbon from the carboxyl end. Sapienate is the most abundant fatty acid in human sebum and among hair-bearing animals is restricted to humans.

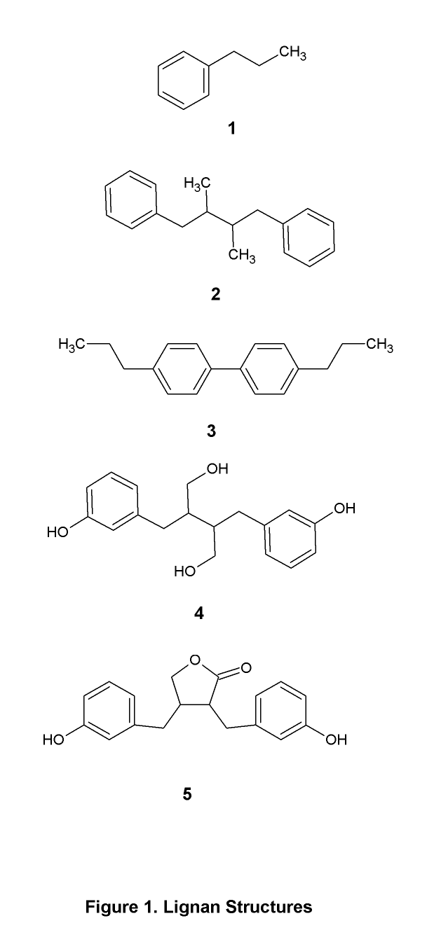

A lignan is a chemical compound found in plants. These natural products are built up of C6C3 units (a propylbenzene skeleton, 1), similar to terpenes, which are built on C5 (isoprene) units. Plant lignans are polyphenolic substances derived from phenylalanine via dimerization of substituted cinnamic alcohols to a dibenzylbutane skeleton 2. A neolignan is skeleton 3. When part of the human diet, lignans are converted into the mammalian lignans known as enterodiol (4) and enterolactone (5) by intestinal bacteria. [FIG. 1].

[FIG. 1].

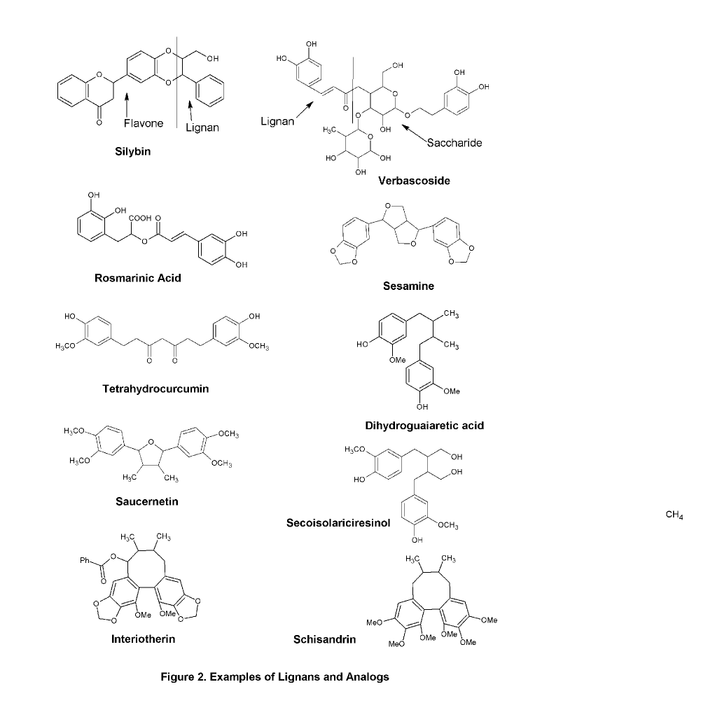

A lignan can be fused to another chemical structure, for example, a flavone, or a saccharide. Biosynthesis of lignans and lignins has been disclosed [Lignin and Lignan Biosynthesis, Lewis et al. (ed), Am. Chem. Soc. Symposium (1998)]. Examples of various lignan structures of importance to the present invention are shown in [FIG. 2].

[FIG. 2].

Natural lignans have been known for some time. Lignans are phenolic-compounds widely distributed in plants. They can be found in different parts (roots, leafs, stem, seeds, fruits) but mainly in small amounts. In many sources (seeds, fruits), lignans are found as glycosidic conjugates associated with fiber component of plants. The most common dietary sources of mammalian lignan precursors are unrefined grain products. The highest concentrations in edible plants have been found in flaxseed, followed by unrefined grain products, particularly rye. Considerable amounts of lignans are also found in coniferous trees. The type of lignans differs in different species and the amounts of lignans vary in different parts of the trees. The typical lignans in heartwood of Norway spruce (Picea abies) are hydroxymatairesinol (HMR), alpha-conidendrin, alpha-conidendric acid, matairesinol, isolariciresinol, secoisolariciresinol, liovil, picearesinol, lariciresinol and pinoresinol [Ekman R: Distribution of lignans in Norway spruce, Acta Academiae Aboensis, Ser B, 39:1-6 (1979)]. The far most abundant single component of lignans in spruce is HMR, about 60 percent of total lignans, which occurs mainly in unconjugated free form. Plant lignans such as hydroxymatairesinol, matairesinol, lariciresinol and secoisolariciresinol, are converted by gut microflora to mammalian lignans, enterolactone or enterodiol. The mammalian lignans can also be manufactured synthetically [M B Groen and J Leemhius, Tetrahedron Letters 21, 5043, (1980)].

Lignans are known to possess beneficial effects on human health. The health benefits obtained with lignan rich diet are, for example, decreased risk for various cancers and cardiovascular diseases (Adlercreutz (1998) Phytoestrogens and human health, In: Reproductive and Developmental Toxicology (edited by Korach, K.). pp. 299-371, Marcel & Dekker, NY.).

Nam [Mini Rev Med Chem., 8, 945 (2006)] disclosed NF-kappaB inhibitor properties of lignans such as manassantins, saucernetin, and saucerneol.

Kubaneck et al. [Phytochemistry, 54, 281 (2000)] report lignans with anti-feedant properties that includes sauriol, licarin, dihydroguaiaretic acid, saucernetin, and saucerneol.

Choong et al. [British Journal of Pharmacology, 146, 752 (2005)] report four lignans isolated from the bark of Machilus thunbergii (Lauraceae) that protected primary cultures of rat cortical neurons from neurotoxicity induced by glutamate. Among the lignans, meso-dihydroguaiarectic acid and licarin-A significantly attenuated glutamate-induced neurotoxicity when added prior to or right after the excitotoxic glutamate challenge.

Kassuya et al. [Eur J. Pharmacol. 546, 182 (2006] report anti-inflammatory action of lignan Nirenthin isolated from Phyllanthus amarus.

Yamauchi et al. [Biosci Biotechnol Biochem, 70, 1934 (2006] report radical and superoxide scavenging activity of lignan Matairesinol and oxidized Matairesinol.

New lignans from sesame, Sesamum indicum, have been identified as saminol and sesamolactol by Grougnet et al. [J. Agric Food Chem. 54, 7570 (2006)].

Shoeb et al. [Phytochemistry, 67, 2370 (2006)] have reported lignan Americanin from the seeds of Centaurea americana, together with five known lignans, arcriin, arctigenin, matairesinol, and lappaol.

A new lignan, isolariciresinol 9-carboxylic acid, has been identified from Smilax bockii by Xu et al. [Pharmazie, 61, 812 (2006)].

Sea buckthorn (Hippophae rhamnoids) has been known for some time for its beneficial effects. Yang et al. [J. Agric Food Che., 54, 8065 (2006)] have isolated secoisolariciresinol and matairesinol, two lignans identified for the first time in sea buckthorn.

Chen et al. [J. Nat Prod., 69, 1697 (2006)] have reported new lignans, Rubrisandrins A and B, from Schisandra rebriflora, along with known lignans, gomisin and schisanhenol. Some of these showed strong anti-HIV activity.

Luo et al. [Chem Biodivers, 3, 224 (2006)] reported lignan glycosides of isolariciresinol from bark of Walsura yunnanensis with potent antioxidant properties.

Yun et al. [Biol Pharm Bull., 30, 139 (2007)] reported lignans, egonol, masutakeside, and styraxlignolide, from the bark of Styrax japonica. These inhibit cyclooxygenase, TNF-alpha, interleukin-1 beta via down-regulation of NF-kappaB-DNA binding activity.

Park et al. [Chem Pharm Bull., 55, 150 (2007)] report a new lignan, isolappaol, along with known lignans lappaol and diarctigenin from the seeds of Arctium lappa. These strongly inhibited NO production.

Coleman et al. [Org. Lett., 7, 1849 (2005)] reported Interiotherin A, a lignan from the stems of Kadsura interior (Schisandraceae family) that inhibits replication of HIV at nanogram levels.

Jang et al (KR20040092782) report a skin whitening cosmetic composition which contains, as an active ingredient, lignan derivatives which are extracted from Machilus Thunbergii and have excellent inhibiting effect on melanin synthesis.

Kim et al. (KR20040058734) disclose a Machilus thunbergii extract, which induces cell apoptosis. In particular, lignan and lactone compounds are isolated from the Machilus thunbergii extract and used for therapeutics for incurable and chronic diseases and food additives.

Hwang et al. (WO 2005070402) disclose a composition for treating acne using lignan compounds.

Ahn et al. (KR20010036445) disclose a lignan compound having antioxidant activity to low density lipoprotein and a process for separating the compound from Saururus chinensis Baill are provided, which can be effectively used for the prevention and treatment of a circulatory disease such as arteriosclerosis by oxidation of low density lipoprotein.

Park et al. (KR20010026440) disclose an ingestible composition for hangover cures and hepatoprotection containing sesame extracts, vitamin E, maltol and green tea extracts, which shows good inhibition effect on peroxidation of lipid. The composition is comprised of 3-30 wt. percent of sesame extracts containing more than 80 percent of sesame lignan compound, which can also contains assistant ingredients such as vitamin B1, B2, B6, B12, C, pantothenic acid, nicotinamide, folic acid, biotin, Zn, Fe, Ca, Mg, and yeast extracts.

Lee et al. (KR20000026053) disclose a lignan compound for the inhibition of the generation of leucotrienes.

Takekoshi et al. (JP2005289838) disclose a lignan melanogenesis promoter in a skin care preparation composition for external use containing the promoter as the effective component.

Son et al. (WO2004096171) disclose a cosmetic composition comprising the extract of Machilus thunbergii and lignan compounds isolated therefrom having potent inhibitory effect on melanin synthesis. Inventive composition shows the excellent inhibitory activity on cultured B-16 melanoma cell therefore, it can be useful in skin whitening cosmetic composition as a cream, skin, lotion, pack and the like.

Renault et al. (WO2004010965) disclose cosmetic use of at least one lignan of given formula, or of a plant extract containing it (such as an extract of flax seeds), in a composition suitable for topical application to the skin, containing a cosmetically acceptable medium, for preventing or treating the signs of ageing of the skin, in particular the loss of firmness, elasticity and/or tonicity of the skin and/or the formation of wrinkles and fine lines. Renault invention also relates to a topical cosmetic composition containing these lignans in combination with other anti-ageing active agents. Lastly, Renault invention relates to a cosmetic process for treating dry skin, comprising the topical application to the said skin of a composition containing at least one such lignan in a cosmetically acceptable medium. Renault et al further disclose (WO2004012697) cosmetic use of at least one lignan of given formula, or of a plant extract containing it (such as an extract of flax seeds), in a composition suitable for topical application to the skin and containing a cosmetically acceptable medium, for preventing or treating dryness of the skin, rough skin and/or a dull complexion, and/or for moisturizing or softening the skin. The invention also relates to a cosmetic process for treating dry skin, comprising the topical application to the said skin of a composition containing at least one such lignan or a plant extract containing it, in a cosmetically acceptable medium. Renault et al. do not disclose any natural lignans that provide treatment of topical discomforts and ailments including sunburn, radiation burn, thermal burn and blisters, shaving burn, wound, venous cannulation, acne, darkened skin pigmentation, skin wrinkles and hair loss, said treatment having been achieved via topical inhibition of Fatty Acid Desaturases.

Wada et al. (JP2000143546) disclose suppressant capable of suppressing the oxidative decomposition of endogenous or exogenous melatonin in vivo and enhancing the stability and utilization efficiency of melatonin in vivo by including a lignan compound in a sesame seed and an antioxidant.

Sakai et al. (JP11255639) disclose a tyrosinase activity inhibitor that is obtained by including, as active ingredient, lignan derivative and/or norlignan compound having a carbonaceous skeleton inherent in lignan or norlignan, with one or both of the substituted two benzene rings representing 4-substituted resorcinol skeleton, wherein the carbon atom on benzyl site succeeding to the 4-substituted resorcinol skeleton bears no substituent.

Murase et al. (JP10175861) disclose a suppressing agent having excellent NF-kappa B activation suppressing effect and useful as a gene expression controlling agent, anti-AIDS virus agent, etc., by using a specific lignan as an active component.

Ko et al. (U.S. patent application Ser. No. 20070020345) disclose a preparation to treat a condition selected from the group consisting of a heart condition, a liver condition, a kidney condition, a lung condition, a cardiovascular condition, myocardial damage or infarction, coronary heart disease, impaired heart-lung function, cancer, heart failure, ischaemia, viral myocarditis, septic/hemorrhagic shock, liver failure, chronic hepatitis, chronic bronchitis, gastritis, type II diabetes, toxic side effects arising from cancer chemotherapy, aging and age-related diseases, liver failure, heart failure, Alzheimer's disease, Parkinson's disease, dehydration, failure of organs and muscle damage comprising administering to a subject in need thereof an effective amount of a pharmaceutical preparation comprising saponins and (−) Schisandrin B.

Empie et al. (U.S. patent application Ser. No. 20060234948) disclose compositions that include a lignan, and an additional compound such as an isoflavone, a tocopherol, a phytosterol, a polyphenol, a catechin, an anthocyanin, an astaxanthin, or a glucosamine. The compositions can be formulated as a dietary supplement, in tablet, powder or liquid form, or can be incorporated into a food product. Methods of treating various diseases by administering the compositions are also provided.

Azar-ul-Haq et al. [Phytomedicine: International Journal of Phytotherapy & Phytopharmacology, 13, 255 (2006)] reported phytochemical investigation of the methanol extract of Vitex negundo, which afforded eight lignans; negundin A 1, negundin B 2, 6-hydroxy-4-(4-hydroxy-3-methoxy)-3-hydroxymethyl-7-methoxy-3,4-dihydro-2-naphthaledehyde 3, vitrofolal E 4, (+)-lyoniresinol 5, (+)-lyoniresinol-3[alpha]-O-[beta]-D-glucoside 6, (+)-(−)-pinoresinol 7, and (+)-diasyringaresinol 8. The structures of these compounds were elucidated unambiguously by spectroscopic methods including 1D and 2D NMR analysis and also by comparing experimental data with literature data. The tyrosinase inhibitory potency of these compounds has been evaluated and attempts to justify their structure-activity relationships showed the compound 5 to be the most potent (I[C.sub.50]=3.21 [micro]M).

Moon et al. [Biol Pharm Bull., 28, 2176 (2005)] report that ethanol and aqueous extracts of Machilus thunbergii used traditionally for the treatments a wide variety of diseases were screened in vitro for the matrix metalloproteinase (MMP)-9 inhibitor actions. Meso-dihydroguaiaretic acid from the stems bark of Machilus thunbergii showed significant MMP-9 inhibition in human keratinocyte cells cause by ultraviolet irradiation. Moon et al. [Phytother Res., 20, 714 (2006)] further report meso-dihydroguaiaretic acid (MDGA) from the stem bark of M. thunbergii showed a significant inhibition of matrix metalloproteinase (MMP)-1 in primary human fibroblasts by heat shock-induced.

Korte et al. (U.S. patent application Ser. No. 20050169947) disclose a topical formulation which includes a lignan or lignan ester in a dermatologically acceptable vehicle. The formulation can be either a cosmetic formulation or a pharmaceutical formulation. Korte et al. do not disclose topical lignan compositions that provide treatment of topical discomforts and ailments including sunburn, radiation burn, thermal burn and blisters, diaper rash, shaving burn, wound, venous cannulation, acne, darkened skin pigmentation, skin wrinkles and hair loss, said treatment achieved via topical inhibition of Fatty Acid Desaturases.

Unno et al. (U.S. patent application Ser. No. 20020051825) disclose a xanthine oxidase inhibitor having as an effective component an extract from highly safe plants, ten kinds of plant materials were compared for the xanthine oxidase inhibition activity, and in consequence Lagerstroemia speciosa (banaba) was found to have the strongest activity. In addition, it was found that the xanthine oxidase inhibition activity was present in an “crude extract” obtained by extracting banaba with hot water or the like, “resin adsorbed components” obtained by adsorbing the crude extract on a styrene-divinyl benzene synthetic resin or the like, and “organic solvent soluble components” obtained by the partition of the resin adsorbed component between water and an organic solvent. Among them, the “organic solvent soluble components” described above were purified with high performance liquid chromatography to obtain “ellagic acid,” an “ellagic acid derivative,” an “ellagic acid analog compound” and a “lignan,” which were found to have a more superior xanthine oxidase inhibitor activity. Unno et al. invention provides a xanthine oxidase inhibitor having each of these extracted materials as an effective component.

Kim et al. (U.S. patent application Ser. No. 20030050335) disclose certain pharmaceutical preparations containing a dibenzocyclooctane lignan derivative for prevention and treatment of neurodegenerative disease.

Arnaud et al. (U.S. patent application Ser. No. 20060177409) disclose new use of lignans for making cosmetic, pharmaceutical and especially dermatological compounds, used to reduce sebum secretion through topical application. The invention also involves a cosmetic care process consisting of applying lignans, possibly in the form of cosmetic compounds, on the area of body and/or facial skin to reduce sebum secretion. Regarding this invention, the compounds are used in particular for the care and/or topic treatment of the body and/or face. However, Arnaud invention pertains to the cosmetic use of lignans, which, according to their claim 10, is due to the inhibition of Type-15-alpha-reductase. This is surprising since the present invention discloses inhibition of fatty acid desaturases by similar lignans.

Kadota et al. (U.S. patent application Ser. No. 20060035964) disclose drugs containing Taxiresinol, (7′R)-7′-Hydroxylariciresinol, Secoisolariciresinol and Isotaxiresinol, which are lignans contained in Hongdoushan, as the active ingredients. Drugs containing an extract, which is obtained by extracting a Hongdoushan plant with water and further extracting the obtained extract with an organic solvent, as the active ingredient. These drugs are useful particularly as a hypoglycemic agent, a liver protecting agent and an anticancer agent.

Silva et al. (WO2006113981) disclose a process to obtain dibenzylbutyrolactonic, tetrahydrofuranic lignans and their synthetic and semi-synthetic derivatives, their analgesic and anti-inflammatory activities, topical and/or systemic formulations containing said lignans and their respective therapeutic method The present invention refers to a process to obtain dibenzylbutyrolactonic lignans from (−)-cubebin, isolated from a Piperaceae, especially Piper cubeba, and from (−)-methylpluviatolide, isolated from a Rutacea, especially Zanthoxylum naranjillo; their synthetic and semi-synthetic derivatives and tetrahydrofuranic lignans, such as galgravin and veragensin, isolated from Nectandra megapotamica, as well as the analgesic and anti-inflammatory activities of said lignans, and the topical and/or systemic formulations in which lignans represent 60 to 80 percent of the formulation. The invention also refers to a therapeutic method using topic and/or systemic formulations based on said lignans for the treatment of inflammation and/or pain.

WO2006067865 (Takagaki et al.) discloses a food capable of in-vivo antioxidant potency enhancement, comprising a proanthocyanidin and a lipid-soluble antioxidant. Preferably, astaxanthin, ubiquinone, lignans, curcumin or a curcumin derivative is used as the lipid-soluble antioxidant. This food capable of in-vivo antioxidant potency enhancement exhibits an excellent antioxidant activity in living body.

WO2006048339 (Burnier et al.) discloses a composition comprising a hydroxylated urea compound and an active agent chosen from dermal relaxant active agents, agents that stimu late the synthesis of dermal or epidermal extracellular matrix fibres or of fibres located at the dermal-epidermal junction and/or that prevent their degradation, antiglycation agents, lipolytic active agents or active agents that inhibit lipogenesis, anti-irritants, and mixtures thereof. The invention also relates to a composition comprising a hydroxylated urea compound and an active agent chosen from carotenoids, retinoids, flavanones, flavonols, isoflavones, coumarins, lignans, stilbenoids, sapogenins, pentacyclic triterpenic acids, ss-hydroxy acids, hydroxyphenols and their ether or heteroside derivatives, phenolic acids, monomers that are tanin precursors, amino sugars, peptides, and mixtures thereof. Application to skincare and to making up the skin.

CN1733131 (Wang et al.) discloses a Schisandra fruit extractive containing at least 50 percent lignans, its preparing process and the use of the extract in preparing medicaments for treating hepatitis, nervous prostration and antisenescence.

Rao et al. (U.S. Pat. No. 6,489,514) disclose a pharmaceutical composition comprising an effective amount of (−)-Secoisolariciresinol together with or associated with an additive useful as an antioxidant; a process for isolating the (−)-Secoisolariciresinol from the plant Stereospermum Personatum and also relates to use of the active fraction (−)-Secoisolariciresinol as an antioxidant or free radical scavenger.

Yao et al. (CN1634905) disclose use of lignans compound used for anti-osteoporosis medicine. The stem and branch of elderberry are heated and refluxed by alcohol and extracted to obtain alcohol extractive, alcohol extractive is mixed and suspended in water and extracted by chloroform, ethyl acetate, and n-butyl alcohol, chloroform extract and ethyl acetate extract are separated by chemical method to obtain lignans compounds carolignan L, carolignan M, and buddlenol G for treating osteoporosis.

Hasselwander et al. (WO2005048970) disclose use of lignans selected from the group comprising secoisolariciresinol, matairesinol, enterolacton, enterodiol, pinoresinol, syringaresinol, isolariciresinol and lariciresinol and glycosides thereof in cosmetic and dermatological preparations.

The above prior art examples illustrate that lignans in general are biologically active. However, most of their biological benefits appear to have been derived via oral administration. None of the above prior art methods disclose any treatments of topical discomforts and ailments including sunburn, radiation burn, thermal burn and blisters, shaving burn, wound, venous cannulation, acne, darkened skin pigmentation, skin wrinkles and hair loss, either via fatty acid desaturase inhibition, or via lignans. However, it should be obvious to any one versed in this art to develop such methodology, as shall be evident from the following discussion.

An omega-3 desaturase that is implicated in the biosynthesis of eicosapentaenoic acid has recently been identified [Pereira et al., Biochem. J., 378 (665(2004)]. This enzyme is unique because it desaturates 20-carbon n-6 fatty acids exclusively, with a preference for arachidonic acid, into eicosapentaenoic acid (20:5, n-3).

Stewart et al. [J. Invest. Dermat. 87, 733 (1986)] reported sebaceous wax esters and epidermal acylceramides that were isolated from skin surface lipid obtained from children and from young adults. Fatty acid methyl esters (FAME) were prepared from the esterified fatty acids of these lipid classes and analyzed to ascertain the proportions of methyl linoleate (18:2 Delta 9,12), methyl sebaleate (18:2 Delta 5,8), and methyl sapienate (16:1 Delta 6). On the same subjects, 2 measures of sebum secretion rate were obtained, namely the sustainable wax ester secretion rate (WESR) on the forehead and the ratio of wax esters/(cholesterol+cholesterol esters) [WE/(CH+CE)] in the surface lipid. The proportions of methyl linoleate in FAME from the wax esters decreased, and the proportions of methyl sebaleate increased, with increased rates of sebum secretion. For both methyl linoleate and methyl sebaleate, a better correlation was obtained when the ratio of WE/(CH+CE) was used as a measure of sebum secretion rather than the WESR. The proportions of methyl linoleate in the FAME from the acylceramides were also inversely related to ratios of WE/(CH+CE). In acylceramides, linoleate was replaced by sapienate, a major fatty acid of human sebum. It appears, therefore, that sebum fatty acid composition may change with changes in sebaceous gland activity, and that sebum fatty acids can enter the epidermis and be incorporated into epidermal lipids.

Yamamoto et al. [J. Invest. Dermat., 89, 507 (1987)], using fused-silica capillary gas chromatography, investigated sebum samples from 55 healthy individuals to discover the effects of aging on the sebaceous gland activity and on the fatty acid composition of wax esters. The sebaceous gland activity, which was expressed by the ratio of wax esters/[cholesterol+cholesterol esters] (WE/[C+CE]), showed a distinct change from infancy through maturity to senescence; the curve of the ratio made a peak in our subjects's 20s. Using the fatty acid analyses, we found an interesting relationship between C16:1 straight and C16:1 iso-branched chains, each of which occupied a large proportion in the fatty acids of wax esters; the former increased in proportion from infancy toward the 20s, with a correlation with aging (r=0.788, p less than 0.01), and decreased thereafter until our subject's 50s (r=−0.611, p less than 0.01). In contrast, the proportion of the latter followed an entirely reversed course with advancing age. The percentages of C16:1 straight chain components were correlated positively with the WE/[C+CE] ratio (r=0.642, p less than 0.01), while there was found to be a negative correlation between the proportion of C16:1 iso-branched chain components and the WE/[C+CE] ratio (r=−0.556, p less than 0.01). The results suggest that more active sebaceous glands in lipid production excrete lipids with a higher proportion of C16:1 straight chain fatty acid and a lower proportion of C16:1 iso-branched chain fatty acid. As well as the sebaceous gland activity, the fatty acid composition in sebum wax esters is affected by advancing age.

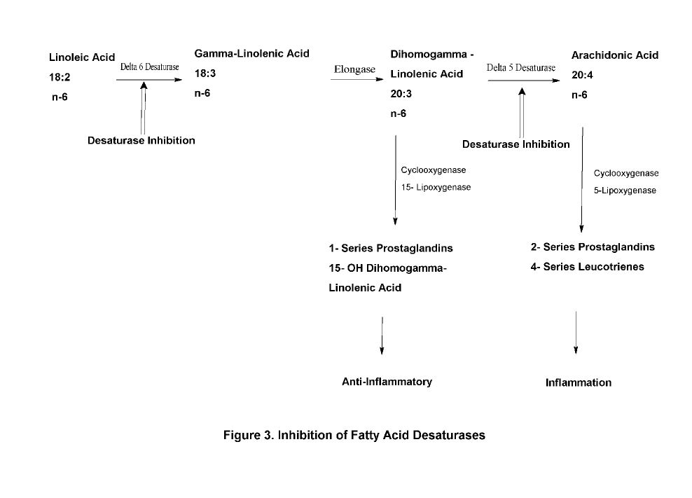

Obukowicz et al. [Pharmacology and Experimental Therapeutics, 287, 157 (1998)] have disclosed Delta-6 or Delta-5 fatty acid desaturase inhibitors as anti-inflammatory agents. In this very important work, decreased synthesis of arachidonic acid, which is a precursor to inflammatory series-2 prostaglandins, as a means to mitigate inflammation was investigated. Arachidonic acid is synthesized from linoleic acid by a sequential series of enzymatic conversions occurring principally in the liver. Specifically, linoleic acid is converted to gamma-linolenic acid (18:3, n-6) by the delta6 desaturase, after which gamma-linolenic acid is elongated to dihomogammalinolenic acid (DGLA, 20:3, n-6) by four discreet enzymes collectively called elongase. DGLA is converted to arachidonic acid by delta5 desaturase [FIG. 3].

[FIG. 3].

Alleviation of inflammation might be attained in humans by decreasing the synthesis of arachidonic acid via selective inhibition of delta6 or delta5 desaturase. Inhibition of the synthesis of arachidonic acid could potentially go beyond amelioration of symptoms currently provided by NSAIDs and EFAD.

It should thus be obvious to develop fatty acid desaturase inhibitors to provide treatment for many topical disorders. Some of such prior art methods are discussed below.

U.S. Pat. No. 5,336,496 (Akimoto et al.) discloses inhibitors for Delta-5-desaturase, including a lignan, for the treatment of inflammation, thrombosis or hypertension. However, Akimoto et al. do not disclose treatment of topical discomforts and ailments including sunburn, radiation burn, thermal burn and blisters, shaving burn, wound, venous cannulation, acne, darkened skin pigmentation, skin wrinkles and hair loss with lignans.

Miyata et al. (U.S. patent application Ser. No. 20060233738) disclose a composition that promotes the production of type I collagen and/or elastin in the human skin fibroblast cells, wherein the composition improves the suppleness and elasticity of the skin, is amply effective in preventing and improving wrinkles and sagging, and is also very safe to the skin. Miyata invention relates to a composition that contains silymarin, which is a general term for flavonolignans such as silybin, silydianin, silychristin and isosilybin, wherein the aforementioned composition has a property to promote the production of type I collagen and/or property to promote the production of elastin. It also relates to a composition containing silymarin derived from a silymarin-containing plant and/or extract of such plant, wherein the aforementioned composition also has a property to promote the production of type I collagen and/or property to promote the production of elastin. However, Miyata et al. do not disclose treatment of topical discomforts and ailments including sunburn, radiation burn, thermal burn and blisters, shaving burn, wound, venous cannulation, acne, darkened skin pigmentation, skin wrinkles and hair loss with lignans.

It is thus known that lignans possess interesting biological benefits, for example, Lignans (Chemistry and Pharmacology of Natural Products), Ayres et al. (2005); Tyrosinase inhibitory lignans from the methanol extract of the roots of Vitex negundo and their structure-activity relationship, Azhar-ul-Haq et al., [Phytomedicine: International Journal of Phytotherapy & Phytopharmacology, 13, 255 (2006)], which relate to their ability to inhibit fatty acid desaturase. Topical Fatty acid desaturases have recently been reported. The topical inhibition of said desaturases by lignans to provide topical treatments should thus be obvious to anyone versed in this art. This has not been achieved so far, despite its obviousness, to provide treatment of topical discomforts and ailments including sunburn, radiation burn, thermal burn and blisters, shaving burn, wound, venous cannulation, acne, darkened skin pigmentation, skin wrinkles, dandruff, and hair loss; said treatment having been achieved via topical inhibition of Fatty Acid Desaturases. This is because said lignans need to be delivered at the dermis, the living site of skin where said desaturases are most active, without being carried away by the blood vessels that are in the epidermis.

A number of topical delivery systems are now known, for example, Gupta (U.S. patent application Ser. No. 20060110415); Date et al. [Skin Pharmacology and Physiology, 19, 2 (2006)]; Skov et al., [J. Pharmaceutical Sci., 86, 1138 (1997)]; and Sentjurk et al., [J. Control Release, 59, 87 (1999)]. To illustrate this complex problem, Date et al. disclose that acne is the most common cutaneous disorder of multifactorial origin with a prevalence of 70-85 percent in adolescents. The majority of the acne sufferers exhibit mild to moderate acne initially, which progresses to the severe form in certain cases. Topical therapy is employed as first-line treatment in mild acne, whereas for moderate and severe acne, systemic therapy is required in addition to topical therapy. Currently, several topical agents are available that affect at least one of the main pathogenetic factors responsible for the development of acne. Although topical therapy has an important position in acne treatment, side effects associated with various topical antiacne agents and the undesirable physicochemical characteristics of certain important agents like tretinoin and benzoyl peroxide affect their utility and patient compliance. Novel drug delivery strategies can play a pivotal role in improving the topical delivery of antiacne agents by enhancing their dermal localization with a concomitant reduction in their side effects. Date et al. emphasize the potential of various novel drug delivery strategies like liposomes, niosomes, aspasomes, microsponges, microemulsions, hydrogels and solid lipid nanoparticles in optimizing and enhancing the topical delivery of antiacne agents, none of which appear to offer a perfect solution. Additional prior art references relative to complexities of topical delivery systems include: Skin Delivery Systems, Wille (ed), Blackwell Publishing (2006) ISBN 0-8138-0848-2; Delivery System Handbook for Personal Care and Cosmetic Products, Meyer (ed), ISBN: 0-8155-1504-9 (2005); Percutaneous Absorption, Maibach (ed) (2005); and Mechanisms of Transdermal Drug Delivery, Potts (1997).

In a surprising and unexpected discovery, it has now been found that certain lignans, when applied topically in combination with a novel method of dermal penetration, provide treatment of topical discomforts and ailments including sunburn, radiation burn, thermal burn and blisters, shaving burn, wound, venous cannulation, acne, darkened skin pigmentation, skin wrinkles and hair loss. The method for the topical delivery of the lignans of the present invention includes the following two steps; (i) the preparation of a penetration composition by combination of said lignan with a solubilizing-penetration enhancing agent in such a ratio as to provide a solubility factor of between 0.1 to 9, preferably between 4 and 6, and (ii) the application of said penetration enhancing composition to the afflicted area of skin where topical benefits are desired in a quantity sufficient to achieve said topical benefits, and (iii) repeating of steps (i) and (ii) until the desired benefits are achieved.

The Preparation of Penetration Composition. A lignan claimed in the present invention is solubilized in a solubilizing agent that has a second role as a penetration enhancing agent. The examples of such solubilizing-penetration enhancing agent include a number of hydroxy acid esters, for example, alkyl and aryl esters of Glycolic Acid, alkyl and aryl esters of Malic Acid, alkyl and aryl esters of Lactic Acid, alkyl and aryl esters of Mandelic Acid, alkyl and aryl esters of Ascorbic Acid, alkyl and aryl esters of Phytic Acid, alkyl and aryl esters of Salicylic Acid, alkyl and aryl esters of Aleuritic Acid, alkyl and aryl esters of Tartaric Acid, alkyl and aryl esters of Citric Acid, alkyl and aryl esters of Hydroxytetronic Acid, alkyl and aryl esters of hydroxycitric acid, alkyl and aryl esters of Glucuronic Acid, alkyl and aryl esters of Hyaluronic Acid, alkyl and aryl esters of Mucic Acid, alkyl and aryl esters of Galacturonic Acid, alkyl and aryl esters of Gluconic Acid, alkyl and aryl esters of Saccharic Acid, alkyl and aryl esters of Glucoheptonic Acid, alkyl and aryl esters of alpha-Hydroxybutyric Acid, alkyl and aryl esters of Hydroxystearic acid, Trihydroxystearin esters, alkyl and aryl esters of Tartronic Acid, alkyl and aryl esters of alpha-Hydroxyisobutyric Acid, alkyl and aryl esters of Isocitric Acid, alkyl and aryl esters of alpha-Hydroxyisocaproic Acid, alkyl and aryl esters of Dihydroxymaleic Acid, alkyl and aryl esters of alpha-Hydroxyisovaleric Acid, alkyl and aryl esters of Dihydroxytartaric Acid, alkyl and aryl esters of beta-Hydroxybutyric Acid, alkyl and aryl esters of Dihydroxyfumaric Acid, alkyl and aryl esters of beta-Phenyllactic Acid, alkyl and aryl esters of Atrolactic Acid, alkyl and aryl esters of Galactonic Acid, alkyl and aryl esters of Pantoic Acid, alkyl and aryl esters of Glyceric Acid. Specific examples of such esters include methyl lactate, ethyl lactate, propyl lactate, isopropyl lactate, butyl lactate, isobutyl lactate, t-butyl lactate, pentyl lactate, neopentyl lactate, isopentyl lactate, hexyl lactate, ethylhexyl lactate, glycerol lactate, benzyl lactate, triethyl citrate, trimethyl citrate, tributyl citrate, acetyl triethyl citrate, acetyl tributyl citrate, trihexyl citrate, butyl trihexyl citrate, stearyl citrate, diethyl tartrate, dimethyl tartrate, ethyl mandelate, ethyl salicylate, methyl salicylate, ethyl glycolate, and combinations thereof. It is both surprising and unexpected that the solubilizing-penetration enhancing agents provide topical penetration exactly to the dermal layers where hair bulb and sebaceous glands are located. Also surprisingly, this solubilizing-penetration enhancing phenomenon has only been observed with the lignans, when said lignans are solubilized and applied topically according to the method of the present invention.

The Determination of Solubility Factor. The lignan is mixed with the solubilizing-penetration enhancing agent and heated at 35 C+−2 C with mixing. The lignan is added until a saturated solution is obtained, as determined by a clear, non-hazy appearance. The ration of lignan to solubilizing-penetration enhancing agent is determined at this point. A solubility factor of from 1 to 9 is acceptable. A solubility factor of 0.1 means 0.1 part of lignan provides a saturated solution in 99.9 parts of solubilizing-penetration enhancing agent. A solubility factor of 9 means 9 parts of lignan provides a saturated solution in 1 part of solubilizing-penetration enhancing agent.

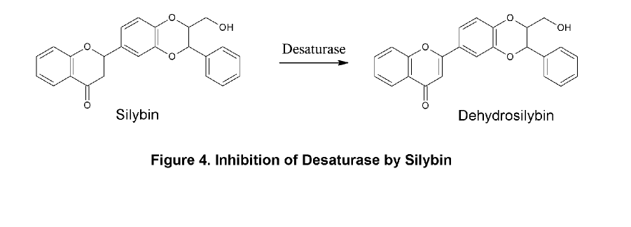

It is also postulated that treatment of topical discomforts and ailments including sunburn, radiation burn, thermal burn and blisters, shaving burn, wound, venous cannulation, acne, darkened skin pigmentation, skin wrinkles, dandruff, and hair loss by the lignans of the present invention is achieved via topical inhibition of Fatty Acid Desaturases by said lignans. The exact mechanism of fatty acid desaturase inhibition by the lignans of the present invention is not clear at this time. It is postulated that such lignans have a saturated two-carbon chain that has two adjacent hydrogen atoms that can be removed with ease. The fatty acid desaturase thus removes those two hydrogen atoms from said lignans instead of removing the same from the fatty acid chain, as further shown in [FIG. 4]. However, this lack of knowledge of exact mechanism of action by lignans of the present invention should not diminish the importance or commercial utility of this invention.

[FIG. 4].

The present invention discloses both unexpected and surprising treatment of topical discomforts and ailments including sunburn, radiation burn, thermal burn and blisters, diaper rash, shaving burn, wound, venous cannulation, acne, darkened skin pigmentation, skin wrinkles, dandruff, and hair loss, said treatment achieved via topical inhibition of Fatty Acid Desaturases.

The present invention also discloses a two-step skin discomfort treatment method comprising; (i) the preparation of a lignan penetration enhancing composition by combination of a lignan, having general chemical structure according to FIG. 1, with a solubilizing-penetration enhancing agent, and (ii) the application of said penetration composition to the afflicted area of skin where topical benefits are desired in a quantity sufficient to achieve desirable topical benefit, and, wherein said solubilizing-penetration enhancing agent causes the penetration of said lignan into the dermis layer of skin, and (iii) repeating of steps (i) and (ii) until the desired benefits are obtained.

In the above treatment method, the lignan is selected from silybin, silymarin, silydianin, silychristin isosilybin, sauriol, licarin, saucernetin, saucerneol, niranthin, Phyllanthin, manassantins, matairesinol, hydroxymatairesinol, oxomatairesinol, saminol, americanin, arctiin, arctigenin, lariciresinol, isolariciresinol, secoisolariciresinol, secoisolariciresinol diglycoside, rubrisandrin, egonol, masutakeside, styraxlignolide, lappaol, diarctigenin, interiotherin, schisandrol, schisandrin, sesamin, sesaminol, episesamin, episesaminol, sesamolin, verbascoside, tetrahydrocurcumin, rosmarinic acid, chlorogenic acid, guaiaretic acid, dihydroguiaretic acid, nor-dihydroguiaretic acid, alpha-conidendrin, liovil, picearesinol, syringaresinol, nortrachelogenin, structural analogs of lignans in [FIG. 1] and [FIG. 2], or combinations thereof.

The said lignans can be derived from a plant and/or extract of such plant.

In the treatment method of the present invention said solubilizing-penetration enhancing agent is selected from the group comprising hydroxy acid esters, which includes methyl lactate, ethyl lactate, propyl lactate, isopropyl lactate, butyl lactate, isobutyl lactate, t-butyl lactate, pentyl lactate, neopentyl lactate, isopentyl lactate, hexyl lactate, ethylhexyl lactate, glycerol lactate, benzyl lactate, triethyl citrate, trimethyl citrate, tributyl citrate, acetyl triethyl citrate, acetyl tributyl citrate, trihexyl citrate, butyl trihexyl citrate, stearyl citrate, diethyl tartrate, dimethyl tartrate, ethyl mandelate, ethyl salicylate, methyl salicylate, ethyl glycolate, and combinations thereof. The said solubilizing-penetration enhancing agent is further selected from the group comprising glycols, which includes glycol ethers, glycol ether esters, glycol ether amides, poly ethylene glycol, polypropylene glycol, polyglycerin, diglycerin, ethoxydiglycol, and methylpropanediol.

An ester of a hydroxy acid can be further selected from the group consisting of:
the methyl, ethyl, propyl, isopropyl, butyl, pentyl, octyl, decyl and dodecyl esters of glycolic acid;
the methyl, ethyl, propyl, isopropyl, butyl, pentyl, octyl, decyl and dodecyl esters of lactic acid;
the methyl, ethyl, propyl, isopropyl, butyl, pentyl, octyl, decyl and dodecyl esters of methyl lactic acid;
the methyl, ethyl, propyl, isopropyl, butyl, pentyl, octyl, decyl and dodecyl esters of 2-hydroxybutanoic acid;
the methyl, ethyl, propyl, isopropyl, butyl, pentyl, octyl, decyl and dodecyl esters of 2-hydroxypentanoic acid; the methyl, ethyl, propyl, isopropyl, butyl, pentyl, octyl, decyl and dodecyl esters of 2-hydroxyhexanoic acid;
the methyl, ethyl, propyl, isopropyl, butyl, pentyl, octyl, decyl and dodecyl esters of 2-hydroxyheptanoic acid;
the methyl, ethyl, propyl, isopropyl, butyl, pentyl, octyl, decyl and dodecyl esters of 2-hydroxyoctanoic acid; the
methyl, ethyl, propyl, isopropyl, butyl, pentyl, octyl, decyl and dodecyl esters of 2-hydroxynonanoic acid;
the methyl, ethyl, propyl, isopropyl, butyl, pentyl, octyl, decyl and dodecyl esters of 2-hydroxydecanoic acid;
the methyl, ethyl, propyl, isopropyl, butyl, pentyl, octyl, decyl and dodecyl esters of 2-hydroxyundecanoic acid;
the methyl, ethyl, propyl, isopropyl, butyl, pentyl, octyl, decyl and dodecyl esters of 2-hydroxydodecanoic acid;
the methyl, ethyl, propyl, isopropyl, butyl, pentyl, octyl, decyl and dodecyl esters of 2-hydroxytetradecanoic acid;
the methyl, ethyl, propyl, isopropyl, butyl, pentyl, octyl, decyl and dodecyl esters of 2-hydroxyhexadecanoic acid;
the methyl, ethyl, propyl, isopropyl, butyl, pentyl, octyl, decyl and dodecyl esters of 2-hydroxyoctadecanoic acid;
the methyl, ethyl, propyl, isopropyl, butyl, pentyl, octyl, decyl and dodecyl esters of 2-hydroxyeicosanoic acid;
the methyl, ethyl, propyl, isopropyl, butyl, pentyl, octyl, decyl and dodecyl esters of 2-hydroxytetraeicosanoic acid; and
the methyl, ethyl, propyl, isopropyl, butyl, pentyl, octyl, decyl and dodecyl esters of 2-hydroxytetraeicosenoic acid, and combinations thereof. The hydroxyacid ester can be an aryl 2-hydroxycarboxylic acid ester further selected from the group consisting of:
the methyl, ethyl, propyl, isopropyl, butyl, pentyl, octyl, decyl and dodecyl esters of 2-phenyl 2-hydroxyethanoic acid esters;
the methyl, ethyl, propyl, isopropyl, butyl, pentyl, octyl, decyl and dodecyl esters of 2,2-diphenyl 2-hydroxyethanoic acid;
the methyl, ethyl, propyl, isopropyl, butyl, pentyl, octyl, decyl and dodecyl esters of 3-phenyl 2-hydroxypropanoic acid; and
the methyl, ethyl, propyl, isopropyl, butyl, pentyl, octyl, decyl and dodecyl esters of 2-phenyl 2-methyl 2-hydroxyethanoic acid, and combinations thereof. The hydroxyacid ester can be an polyhydroxyacid ester selected from the group consisting of:
the methyl, ethyl, propyl, isopropyl, butyl, pentyl, octyl, decyl, dodecyl, phenyl and benzyl esters of 2,3-dihydroxypropanoic acid;
the methyl, ethyl, propyl, isopropyl, butyl, pentyl, octyl, decyl, dodecyl, phenyl and benzyl esters of 2,3,4-trihydroxybutanoic acid and its isomers including erythronic acid and threonic acid; the methyl, ethyl, propyl, isopropyl, butyl, pentyl, octyl, decyl, dodecyl, phenyl and benzyl esters of 2,3,4,5-tetrahydroxypentanoic acid and its isomers including ribonic acid, arabinoic acid, xylonic acid and lyxonic acid;
the methyl, ethyl, propyl, isopropyl, butyl, pentyl, octyl, decyl, dodecyl, phenyl and benzyl esters of 2,3,4,5,6-pentahydroxyhexanoic acid and its isomers including allonic acid, altronic acid, gluconic acid, mannoic acid, gulonic acid, idonic acid, galactonic acid, and talonic acid; the methyl, ethyl, propyl, isopropyl, butyl, pentyl, octyl, decyl, dodecyl, phenyl and benzyl esters of 2,3,4,5,6,7-hexahydroxyheptanoic acid and its isomers including glucoheptonic acid and galactoheptonic acid;
the methyl, ethyl, propyl, isopropyl, butyl, pentyl, octyl, decyl, dodecyl, phenyl and benzyl esters of glyceruronic acid;
the methyl, ethyl, propyl, isopropyl, butyl, pentyl, octyl, decyl, dodecyl, phenyl and benzyl esters of erythruronic acid;
the methyl, ethyl, propyl, isopropyl, butyl, pentyl, octyl, decyl, dodecyl, phenyl and benzyl esters of threuronic acid;
the methyl, ethyl, propyl, isopropyl, butyl, pentyl, octyl, decyl, dodecyl, phenyl and benzyl esters of riburonic acid;
the methyl, ethyl, propyl, isopropyl, butyl, pentyl, octyl, decyl, dodecyl, phenyl and benzyl esters of arabinuronic acid;
the methyl, ethyl, propyl, isopropyl, butyl, pentyl, octyl, decyl, dodecyl, phenyl and benzyl esters of xyluronic acid;
the methyl, ethyl, propyl, isopropyl, butyl, pentyl, octyl, decyl, dodecyl, phenyl and benzyl esters of lyxuronic acid;
the methyl, ethyl, propyl, isopropyl, butyl, pentyl, octyl, decyl, dodecyl, phenyl and benzyl esters of alluronic acid;
the methyl, ethyl, propyl, isopropyl, butyl, pentyl, octyl, decyl, dodecyl, phenyl and benzyl esters of altruronic acid;
the methyl, ethyl, propyl, isopropyl, butyl, pentyl, octyl, decyl, dodecyl, phenyl and benzyl esters of glucuronic acid;
the methyl, ethyl, propyl, isopropyl, butyl, pentyl, octyl, decyl, dodecyl, phenyl and benzyl esters of mannuronic acid;
the methyl, ethyl, propyl, isopropyl, butyl, pentyl, octyl, decyl, dodecyl, phenyl and benzyl esters of guluronic acid;
the methyl, ethyl, propyl, isopropyl, butyl, pentyl, octyl, decyl, dodecyl, phenyl and benzyl esters of iduronic acid;
the methyl, ethyl, propyl, isopropyl, butyl, pentyl, octyl, decyl, dodecyl, phenyl and benzyl esters of galacturonic acid; and
the methyl, ethyl, propyl, isopropyl, butyl, pentyl, octyl, decyl, dodecyl, phenyl and benzyl esters of taluronic acid, and combinations thereof. The hydroxyacid ester can be a hydroxypolyacid ester further selected from the group consisting of:
the methyl, ethyl, propyl, isopropyl, butyl, pentyl, octyl, decyl, dodecyl, phenyl and benzyl esters of 2-hydroxypropane-1,3-dioic acid;
the methyl, ethyl, propyl, isopropyl, butyl, pentyl, octyl, decyl, dodecyl, phenyl, benzyl, dimethyl, diethyl, dipropyl, diisopropyl, dibutyl, dipentyl, dioctyl, didecyl, didodecyl, diphenyl and dibenzyl esters of 2-hydroxybutane-1,4-dioic acid esters;
the methyl, ethyl, propyl, isopropyl, butyl, pentyl, octyl, decyl, dodecyl, phenyl, benzyl, dimethyl, diethyl, dipropyl, diisopropyl, dibutyl, dipentyl, dioctyl, didecyl, didodecyl, diphenyl and dibenzyl esters of 2,3-dihydroxybutane-1,4-dioic acid; and
the methyl, ethyl, propyl, isopropyl, butyl, pentyl, octyl, decyl, dodecyl, phenyl, benzyl, dimethyl, diethyl, dipropyl, diisopropyl, dibutyl, dipentyl, dioctyl, didecyl, didodecyl, diphenyl, dibenzyl, trimethyl, triethyl, tripropyl, triisopropyl, tributyl, tripentyl, trioctyl, tridecyl, tridodecyl, triphenyl and tribenzyl esters of 3-hydroxy-3-carboxypentane-1,5-dioic acid, and combinations thereof.

The present invention also discloses a method for topical inhibition of Fatty Acid Desaturase, comprising (i) the mixing of a lignan with a solubilizing penetration enhancing agent, and (ii) the application of said mixture on skin in an effective amount, and (iii) wherein said carrier causes the penetration of said lignan into the dermis layer of skin, and said lignan causes the inhibition of said Fatty Acid Desaturase therein; and wherein said method is for the reduction of skin wrinkles, skin brightening, reduction of eyelid puffiness, or the control of oily skin including oily skin from acne, and similar topical discomforts.

The amount of lignan, which can be used according to the present invention, depends of course on the desired effect and can therefore vary within a large range, this amount being within the skill of the ordinary artisan in view of this disclosure. To give an order of magnitude, the lignans can be used in an amount representing from 0.0001 percent to 5.0 percent of the total weight of a composition, preferably in an amount representing from 0.01 percent to 2.0 percent of the total weight of the composition. The plant extract containing a lignan can be used in an amount representing from 0.0001 percent to 20 percent of the total weight of a composition, depending on percent solids content of the plant extract and the amount and nature of the extraction solvent present in such extract.

Synergistic benefits are noted when a Citrate Lyase enzyme inhibitor agent is included in combination with lignans of the present invention. The examples of such agents include Forskohlin, Coleus forskohlii extract, Momordica Charantia extract, Charantins, Momordicosides, Hydroxycitric acid, Garcinia cambogia extract, Phaseolamin, Phaseolus vulgaris extract, Synephrine, Hordenine, Octopamine, Tyramine, n-Methyltyramine, and combinations thereof.

Synergistic benefits are also noted when an additional agent known to impart complimentary benefits is included. The examples of such agents are known anti-acne agents including salicylic acid, benzoyl peroxide, resorcinol, zinc salicylate, hydroxy acids, and zinc amino acid complexes of hydroxy acids.

Synergistic benefits are also noted when an additional agent known to impart complimentary wound healing benefits by stimulating collagen and elastin synthesis are included.

Control of Acne.

The compositions of the present invention provide an unexpected and previously unknown treatment for acne. This is discussed further as noted below.

The oil on the surface of skin is a complex mixture of sebum lipids (from the surface skin cells), sweat and environmental material. Sebum is produced by sebaceous glands. These are found over most of the body including head, although there are few on the hands or feet and none on the palms and soles. Sebaceous glands on the mid-back, forehead and chin are larger and more numerous than elsewhere (up to 400-900 glands per square centimetre). They are also numerous in the ear canal and around the genitals. The sebaceous gland consists of lobes connected by ducts, which are lined with cells similar to those on the skin surface. The sebum flow dynamics at the skin surface results from a multi-step process starting with sebocyte proliferation, intracellular lipid synthesis, cell lysis in the sebaceous duct, storage of sebum in the follicular reservoir, discharge through the follicular opening and spreading over the stratum corneum [Pierard, Dermatology, vol. 196, pages 126-129 (1998)]. Most sebaceous glands open out into the hair follicle. Some free sebaceous glands open directly onto the skin surface. These include Meibomian glands on the eyelids, Tysons glands on the foreskin and Fordyces spots on the upper lip. Sebum is produced when the sebaceous gland disintegrates. The cells take about a week from formation to discharge. Sebum is a complex and variable mixture of lipids including: Glycerides, Free fatty acids, Wax esters, Squalene, Cholesterol esters, and Cholesterol [Stewart, M. E., Semin. Dermatol. 11, 100-105 (1992)]. The action of bacterial lipases converts a varying portion of sebum triglycerides to free fatty acids, which are known to cause inflammation and skin discomforts including acne and hair loss.

The sebocyte constitutes the competent cell of the sebaceous gland. The production of sebum is associated with the program of terminal differentiation of this cell. During this differentiation, the metabolic activity of the sebocyte is essentially centered around the biosynthesis of lipids (lipogenesis), and more precisely on the neosynthesis of fatty acids and the squalene. A compound making it possible to reduce the production of the lipids constituting sebum, by the cells of the sebaceous gland (sebocytes), would therefore be of definite value for the treatment of oily skin. It will also be useful for the reduction of excess skin oil, for example, from acne.

Lan et al. [Journal of Investigative Dermatology, 120, 707 (2003)] have recently shown that Delta-6-saturase, also known as fatty acid desaturase-2, is a component of a lipid metabolic pathway that converts the essential fatty acids linoleate and alpha-linoleate into long-chain polyunsaturated fatty acids. This enzyme has previously been known only in the internal organs, such as liver and spleen, and not in skin. Lan et al. also isolated Delta-6-desaturase from skin cells, including scalp. Moreover, within human skin, Delta-6-desaturase was found to have been restricted to differentiating sebocytess located in the suprabasal layers of the sebaceous gland. Enzymatic analysis using CHO cells overexpressing human Delta-6-desaturase indicates catalysis of a polyunsaturated fatty acid type reaction, but also an unexpected sebaceous type reaction; that of converting palmitate into the mono-unsaturated fatty acid, sapienate, a 16-carbon fatty acid with a single cis-double bond at the sixth carbon atom from the carboxyl end. Sapienate is the most abundant fatty acid on human sebum, and among hair bearing animals is restricted to humans. Thus, Lan et al. have shown, for the first time, Delta-6-saturase as the major fatty acid desaturase in human sebaceous glands, and suggests that the environment of the sebaceous gland permits catalysis of the sebaceous-type reaction and restricts catalysis of the polyunsaturated fatty acid type reaction. Two additional reports have appeared concerning Delta-6 desaturase. M. Mizaki and coworkers (J. Lipid Res. 43:2146, 2002) reported an unspecified desaturase enzyme activity able to synthesize sapienate in mouse preputial glands, a sebaceous gland of the urogenital tract. Subsequently, H. Guillou and coworkers (J. Lipid Res. 44:450, 2003) showed that COS-7 cells transfected with a rat liver FADS2 cDNA acquired the ability to desaturate both palmitic and linoleic acid at the 6 position. This restriction of sapienate synthesis to a rodent sexual organ supports a role for human sebaceous gland-derived sapienate in sexual maturation. These findings have not been utilized in the prior art for the treatment of skin conditions related to sapienate production via the inhibition of topical Delta-6-desaturate. It is a further objective of the present invention to provide this solution. The compositions of the present invention provide an unexpected and previously unknown treatment for acne.

Control of Dark Spots. Inhibition of Phenylalanine Hydroxylase and Phenylalanine Transaminase to Cause Skin Brightening.

Phenylalanine hydroxylase is responsible for the first step in the conversion of phenylalanine into tyrosine. Tyrosine is required for the production of melanin, which gives color to hair and skin. Phenylalanine hydroxylase must work in combination with tetrahydrobiopterin to perform this function. Phenylalanine hydroxylase contains iron in its active site, and tetrahydrobiopterin is required in proximity to this active site.

It is both surprising and unexpected that lignan compositions of the present invention inhibit phenylalanine hydroxylase, resulting in skin brightening affect. In the biosynthesis of lignans, Phenylalanine is converted into trans-cinnamic acid by Phenylalanine ammonia-lyase (PAL; EC 4.3.1.5), which is then converted into an activated hydroxycinnamic acid via the action of Cinnamate 4-hydroxylase (CH4; EC 1.14.13.11), as discussed by Achnine et al. [The Plant Cell, 16, 3098 (2004)]. Although the mechanism of phenylalanine hydroxylase inhibition by the lignans of the present invention is not fully clear at this time, it is theorized that phenylalanine hydroxylase may be diverted to said lignans thus causing the shutdown of the conversion of phenylalanine into tyrosine. This could be the cause for the skin brightening effect. The lignans of the present invention thus do not seem to directly inhibit tyrosinase, as claimed in the prior art citations mentioned elsewhere in the present disclosure. The net effect of skin brightening by lignans of the present invention is thus both unexpected and unprecedented.

Anti-Wrinkle—Antiaging Applications.

The compositions of the present invention provide an unexpected inhibition of inflammatory prostaglandin biosynthesis. This results in an anti-inflammatory effect, which, via a cascade mechanism, increases suppleness of skin, leading to reduced visible skin winkles from aging. The exact biochemical mechanism for these unexpected benefits is not yet known.

Wound and Burn Healing Applications.

The compositions of the present invention provide an unexpected wound healing benefit with little scar tissue formation or skin pigment discoloration.

The entire wound healing process is a complex series of events that begins at the moment of injury and can continue for months to years. This overview will help in identifying the various stages of wound healing.

I. Inflammatory Phase. A) Immediate to 2-5 days; B) Hemostasis, (i) Vasoconstriction, (ii) Platelet aggregation, and (iii) Thromboplastin makes clot; C) Inflammation, (i) Vasodilation, (ii) Phagocytosis.

II. Proliferative Phase. A) 2 days to 3 weeks; B) Granulation, (i) Fibroblasts lay bed of collagen, (ii) Fills defect and produces new capillaries; C) Contraction, (i) Wound edges pull together to reduce defect; D) Epithelialization, (i) Crosses moist surface, (ii) Cell travel about 3 cm from point of origin in all directions.

III. Remodeling Phase. A) 3 weeks to 2 years; B) New collagen forms which increases tensile strength to wounds; C) Scar tissue is only 80 percent as strong as original tissue.

Wound healing, or wound repair, is the body's natural process of regenerating dermal and epidermal tissue. When an individual is wounded, a set of events takes place in a predictable fashion to repair the damage. These events overlap in time and must be artificially categorized into separate steps: the inflammatory, proliferative, and maturation phases. In the inflammatory phase, bacteria and debris are phagocytized and removed and factors are released that cause the migration and division of cells involved in the proliferative phase. The proliferative phase is characterized by angiogenesis, collagen deposition, granulation tissue formation, epithelialization, and wound contraction. In angiogenesis, new blood vessels grow from endothelial cells. In fibroplasia and granulation tissue formation, fibroblasts grow and form a new, provisional extracellular matrix (ECM) by excreting collagen and fibronectin. In epithelialization, epithelial cells crawl across the wound bed to cover it. In contraction, the wound is made smaller by the action of myofibroblasts, which establish a grip on the wound edges and contract themselves using a mechanism similar to that in smooth muscle cells. When the cells' roles are close to complete, unneeded cells undergo apoptosis. In the maturation and remodeling phase, collagen is remodeled and realigned along tension lines and cells that are no longer needed are removed by apoptosis.

In the inflammatory phase, clotting takes place in order to obtain hemostasis, or stop blood loss, and various factors are released to attract cells that phagocytise debris, bacteria, and damaged tissue and release factors that initiate the proliferative phase of wound healing. When tissue is first wounded, blood comes in contact with collagen, triggering blood platelets to begin secreting inflammatory factors. Platelets also express glycoproteins on their cell membranes that allow them to stick to one another and to aggregate, forming a mass. Fibrin and fibronectin cross-link together and form a plug that traps proteins and particles and prevents further blood loss. This fibrin-fibronectin plug is also the main structural support for the wound until collagen is deposited. Migratory cells use this plug as a matrix to crawl across, and platelets adhere to it and secrete factors. The clot is eventually lysed and replaced with granulation tissue and then later with collagen. Platelets, the cells present in the highest numbers shortly after wounding, release a number of factors into the blood, including ECM proteins and cytokines, including growth factors. Growth factors stimulate cells to speed their rate of division. Platelets also release other proinflammatory factors like serotonin, bradykinin, prostaglandins, prostacyclins, thromboxane, and histamine, which serve a number of purposes, including to increase cell proliferation and migration to the area and to cause blood vessels to become dilated and porous.

Contrary to common belief, the use of any anti-inflammatory agents during the early stages of wound-healing process is not desirable. On the same note, the over-expression of 2-series prostaglandins should be controlled from the onset of wound and burn healing process to avoid apoptosis of newly formed connective tissue. Both connective tissue forming agents and anti-inflammatory agents are beneficial during the later stages of wound healing.

Unexpectedly, the lignan agents of the present invention assist in the wound healing mechanism, the exact nature if which is not yet known. However, this should not preclude the practical utility of the present invention in topical wound healing applications.

Dandruff Control.

Dandruff is the result of the normal growing process of the skin cells of the scalp. Shedding of dead skin cells from the scalp at an excessive rate is the result of the normal growing process of the skin cells of the scalp. In a normal scalp, the process of sloughing off old cells and manufacturing of their replacements is very orderly and complete. In the dandruff scalp, there is mass disorder and often the departing cells are not dead before leaving the scalp. Contrary to popular theory, although bacteria may aggravate a dandruff condition, bacteria do not cause the initial problem. Most medical authorities consider dandruff, even the mildest forms, to be a type of scalp or skin related disease. Clinically, one description of dandruff is Seborrhea Capitos or excessive sebum production of the scalp. Today most skin specialists agree that dandruff is associated with a tiny fungus called Pityrosporum ovale, or P. ovale for short. This fungus lives on our bodies and scalp all the time, usually without causing a problem. It has been theorized that P. ovale metabolizes excess oil on scalp, which results in the formation of lower molecular weight fatty acids that cause skin irritation leading to dandruff. Although the present inventor has not studied the biochemical mechanism for the effect of compositions of the present invention on dandruff itself, said compositions of the present invention have been found to reduce the excess sebum or oil on scalp and also reduce dandruff formation.

For topical application to the skin, the lignans of the present invention may be provided in any cosmetic or pharmaceutical form normally used in the cosmetics and dermatological fields, and it may in particular be in the form of an aqueous, optionally gelled, solution, of a dispersion of the optionally two-phase lotion type, of an emulsion obtained by dispersion of a fatty phase (oil) in an aqueous phase (O/W) or vice versa (W/O), of a triple emulsion (W/O/W or O/W/O) or of a vesicular dispersion of the ionic and/or nonionic type. These compositions may be prepared according to the usual methods. This composition may be more or less fluid and have the appearance of a cream, an ointment, a milk, a lotion, a serum, a paste, and a mousse. It may optionally be applied in the form of an aerosol. It may also be provided in solid form, in particular in the form of a stick. It may be used as a care product and/or as a make-up product for the skin. It may also be used as a shampoo or a conditioner.

The lignans of the present invention can be formulated in various cosmetic and pharmaceutical consumer products utilizing a variety of delivery systems and carrier bases. Such consumer product forms include the group consisting of shampoos, aftershaves, sunscreens, body and hand lotions, skin creams, liquid soaps, bar soaps, bath oil bars, shaving creams, conditioners, permanent waves, hair relaxers, hair bleaches, hair detangling lotion, styling gel, styling glazes, spray foams, styling creams, styling waxes, styling lotions, mousses, spray gels, pomades, shower gels, bubble baths, hair coloring preparations, conditioners, hair lighteners, coloring and non-coloring hair rinses, hair grooming aids, hair tonics, spritzes, styling waxes, band-aids, and balms.

In another preferred aspect, the delivery system can be traditional water and oil emulsions, suspensions, colloids, micro emulsions, clear solutions, suspensions of nanoparticles, emulsions of nanoparticles, or anhydrous compositions.

The compositions of the present invention may also contain adjuvants which are used in the cosmetics field, such as hydrophilic or lipophilic gelling agents, hydrophilic or lipophilic active agents, preserving agents, antioxidants, solvents, fragrances, fillers, screening agents, pigments, odor absorbers and dyestuffs. The amounts of these various adjuvants may be those conventionally used in the field considered. These adjuvants, depending on their nature, can be introduced into the fatty phase, into the aqueous phase or into the lipid vesicles. In addition, moisturizers may complete the effect obtained using the sapogenins according to the invention and anti-inflammatory agents are also useful.

The application of skin complexion enhancement agents of the present invention can be in several areas of consumer interest, some of which include control of excess facial oil associated with acne, control of excess oil on scalp associated with dandruff, and control of excess body and underarm oil associated with body and underarm malodor.

Additional cosmetically or pharmaceutically beneficial ingredients can also be included in the compositions of the present invention, which can be selected from skin cleansers, cationic, anionic surfactants, non-ionic surfactants, amphoteric surfactants, and zwitterionic surfactants, skin and hair conditioning agents, vitamins, hormones, minerals, plant extracts, anti-inflammatory agents, collagen and elastin synthesis boosters, UVA/UVB sunscreens, concentrates of plant extracts, emollients, moisturizers, skin protectants, humectants, silicones, skin soothing ingredients, antimicrobial agents, antifungal agents, treatment of skin infections and lesions, blood microcirculation improvement, skin redness reduction benefits, additional moisture absorbents, analgesics, skin penetration enhancers, solubilizers, moisturizers, emollients, anesthetics, colorants, perfumes, preservatives, seeds, broken seed nut shells, silica, clays, beads, luffa particles, polyethylene balls, mica, pH adjusters, processing aids, and combinations thereof.

In another preferred aspect, the cosmetically acceptable composition further comprises one or more excipient selected from the group consisting of water, saccharides, surface active agents, humectants, petrolatum, mineral oil, fatty alcohols, fatty ester emollients, waxes and silicone-containing waxes, silicone oil, silicone fluid, silicone surfactants, volatile hydrocarbon oils, quaternary nitrogen compounds, amine functionalized silicones, conditioning polymers, rheology modifiers, antioxidants, sunscreen active agents, di-long chain amines from about C.sub.10 to C.sub.22, long chain fatty amines from about C.sub.10 to C.sub.22, fatty alcohols, ethoxylated fatty alcohols and di-tail phospholipids.

Representative saccharides include nonionic or cationic saccharides such as agarose, amylopectins, amyloses, arabinans, arabinogalactans, arabinoxylans, carageenans, gum arabic, carboxymethyl guar gum, carboxymethyl(hydroxypropyl) guar gum, hydroxyethyl guar gum, carboxymethyl cellulose, cationic guar gum, cellulose ethers including methyl cellulose, chondroitin, chitins, chitosan, chitosan pyrrolidone carboxylate, chitosan glycolate chitosan lactate, cocodimonium hydroxypropyl oxyethyl cellulose, colominic acid ([poly-N acetyl-neuraminic acid]), corn starch, curdlan, dermatin sulfate, dextrans, furcellarans, dextrans, cross-linked dextrans, dextrin, emulsan, ethyl hydroxyethyl cellulose, flaxseed saccharide (acidic), galactoglucomannans, galactomannans, glucomannans, glycogens, guar gum, hydroxy ethyl starch, hydroxypropyl methyl cellulose, hydroxy ethyl cellulose, hydroxy propyl cellulose, hydroxypropyl starch, hydroxypropylated guar gums, gellan gum, gellan, gum ghatti, gum karaya, gum tragancanth (tragacanthin), heparin, hyaluronic acid, inulin, keratin sulfate, konjac mannan, modified starches, laminarans, laurdimonium hydroxypropyl oxyethyl cellulose, okra gum, oxidized starch, pectic acids, pectin, polydextrose, polyquaternium-4, polyquaternium-10, polyquaternium-28, potato starch, protopectins, psyllium seed gum, pullulan, sodium hyaluronate, starch diethylaminoethyl ether, steardimonium hydroxyethyl cellulose, raffinose, rhamsan, tapioca starch, whelan, levan, scleroglucan, sodium alginate, stachylose, succinoglycan, wheat starch, xanthan gum, xylans, xyloglucans, and mixtures thereof. Microbial saccharides can be found in Kirk-Othmer Encyclopedia of Chemical Technology, Fourth Edition, Vol. 16, John Wiley and Sons, NY pp. 578-611 (1994), which is incorporated entirely by reference. Complex carbohydrates found in Kirk-Othmer Encyclopedia of Chemical Technology, Fourth Edition, Vol. 4, John Wiley and Sons, NY pp. 930-948, 1995 which is herein incorporated by reference.

The cosmetically acceptable composition of the present invention may include surface-active agents. Surface-active agents include surfactants, which typically provide detersive functionality to a formulation or act simply as wetting agents. Surface-active agents can generally be categorized as anionic surface-active agents, cationic surface-active agents, nonionic surface-active agents, amphoteric surface-active agents and zwitterionic surface-active agents, and dispersion polymers. Anionic surface-active agents useful herein include those disclosed in U.S. Pat. No. 5,573,709, incorporated herein by reference. Examples include alkyl and alkyl ether sulfates. Specific examples of alkyl ether sulfates which may be used In this invention are sodium and ammonium salts of lauryl sulfate, lauryl ether sulfate, coconut alkyl triethylene glycol ether sulfate; tallow alkyl triethylene glycol ether sulfate, and tallow alkyl hexaoxyethylene sulfate. Highly preferred alkyl ether sulfates are those comprising a mixture of individual compounds, said mixture having an average alkyl chain length of from about 12 to about 16 carbon atoms and an average degree of ethoxylation of from about 1 to about 6 moles of ethylene oxide. Another suitable class of anionic surface-active agents is the alkyl sulfuric acid salts. Important examples are the salts of an organic sulfuric acid reaction product of a hydrocarbon of the methane series, including iso-, neo-, and n-paraffins, having about 8 to about 24 carbon atoms, preferably about 12 to about 18 carbon atoms and a sulfonating agent, for example, sulfur trioxide or oleum, obtained according to known sulfonation methods, including bleaching and hydrolysis. Preferred are alkali metal and ammonium sulfated C.sub.12-38 n-paraffins.

Additional synthetic anionic surface-active agents include the olefin sulfonates, the beta-alkyloxy alkane sulfonates, and the reaction products of fatty acids esterified with isethionic acid and neutralized with sodium hydroxide, as well as succinamates. Specific examples of succinamates include disodium N-octadecyl sulfosuccinamate; tetrasodium N-(1,2-dicarboxyethyl)-N-octadecylsulfosuccinamate; diamyl ester of sodium sulfosuccinic acid; dihexyl ester of sodium sulfosuccinic acid; dioctyl esters of sodium sulfosuccinic acid.

Preferred anionic surface-active agents for use in the cosmetically acceptable composition of present invention include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, and sodium dodecyl benzene sulfonate.

Amphoteric surface-active agents which may be used in the cosmetically acceptable composition of present invention include derivatives of aliphatic secondary and tertiary amines, in which the aliphatic substituent contains from about 8 to 18 carbon atoms and an anionic water solubilizing group e.g., carboxy, sulfonate, sulfate, phosphate, or phosphonate. Representative examples include sodium 3-dodecyl-aminopropionate, sodium 3-dodecylaminopropane sulfonate, sodium lauryl sarcosinate, N-alkyltaurines such as the one prepared by reacting dodecylamine with sodium isethionate as described in U.S. Pat. No. 2,658,072, N-higher alkyl aspartic acids as described in U.S. Pat. No. 2,438,091, and the products sold under the trade name MIRANOL. as described in U.S. Pat. No. 2,528,378. Other sarcosinates and sarcosinate derivatives can be found in the CTFA Cosmetic Ingredient Handbook, Fifth Edition, 1988, page 42 incorporated herein by reference.

Quaternary ammonium compounds can also be used in the cosmetically acceptable composition of the present invention as long as they are compatible in the compositions of the invention, wherein the structure is provided in the CTFA Cosmetic Ingredient Handbook, Fifth Edition, 1988, page 40. Cationic surface-active agents generally include, but are not limited to fatty quaternary ammonium compounds containing from about 8 to about 18 carbon atoms. The anion of the quaternary ammonium compound can be a common ion such as chloride, ethosulfate, methosulfate, acetate, bromide, lactate, nitrate, phosphate, or tosylate and mixtures thereof. The long chain alkyl groups can include additional or replaced carbon or hydrogen atoms or ether linkages. Other substitutions on the quaternary nitrogen can be hydrogen, hydrogen, benzyl or short chain alkyl or hydroxyalkyl groups such as methyl, ethyl, hydroxymethyl or hydroxyethyl, hydroxypropyl or combinations thereof.

Examples of quaternary ammonium compounds include but are not limited to; Behentrimonium chloride, Cocotrimonium chloride, Cethethyldimonium bromide, Dibehenyldimonium chloride, Dihydrogenated tallow benzylmonium chloride, disoyadimonium chloride, Ditallowdimonium chloride, Hydroxycetyl hydroxyethyl dimonium chloride, Hydroxyethyl Behenamidopropyl dimonium chloride, Hydroxyethyl Cetyldimonium chloride, Hydroxyethyl tallowdimonium chloride, myristalkonium chloride, PEG-2 Oleamonium chloride, PEG-5 Stearmonium chloride, PEG-15 cocoyl quaternium 4, PEG-2 stearalkonium 4, lauryltrimonium chloride; Quaternium-16; Quaternium-18, lauralkonium chloride, olealkmonium chloride, cetylpyridinium chloride, Polyquaternium-5, Polyquaternium-6, Polyquaternium-7, Polyquaternium-10, Polyquaternium-22, Polyquaternium-37, Polyquaternium-39, Polyquaternium-47, cetyl trimonium chloride, dilauryldimonium chloride, cetalkonium chloride, dicetyldimonium chloride, soyatrimonium chloride, stearyl octyl dimonium methosulfate, and mixtures thereof. Other quaternary ammonium compounds are listed in the CTFA Cosmetic Ingredient Handbook, First Edition, on pages 41-42, incorporated herein by reference.

The cosmetically acceptable compositions of the present invention may include long chain fatty amines from about C.sub.10 to C.sub.22 and their derivatives. Specific examples include dipalmitylamine, lauramidopropyldimethylamine, and stearamidopropyl dimethylamine. The cosmetically acceptable compositions of this invention may also include fatty alcohols (typically monohydric alcohols), ethoxylated fatty alcohols, and di-tail phospholipids, which can be used to stabilize emulsion or dispersion forms of the cosmetically acceptable compositions. They also provide a cosmetically acceptable viscosity. Selection of the fatty alcohol is not critical, although those alcohols characterized as having fatty chains of C.sub.10 to C.sub.32, preferably C.sub.14 to C.sub.22, which are substantially saturated alkanols will generally be employed. Examples include stearyl alcohol, cetyl alcohol, cetostearyl alcohol, myristyl alcohol, behenyl alcohol, arachidic alcohol, isostearyl alcohol, and isocetyl alcohol. Cetyl alcohol is preferred and may be used alone or in combination with other fatty alcohols, preferably with stearyl alcohol. When used the fatty alcohol is preferably included in the formulations of this invention at a concentration within the range from about 1 to about 8 weight percent, more preferably about 2 to about 6 weight percent. The fatty alcohols may also be ethoxylated. Specific examples include cetereth-20, steareth-20, steareth-21, and mixtures thereof. Phospholipids such as phosphatidylserine and phosphatidylcholine, and mixtures thereof may also be included. When used, the fatty alcohol component is included in the formulations at a concentration of about 1 to about 10 weight percent, more preferably about 2 to about 7 weight percent.

Nonionic surface-active agents, which can be used in the cosmetically acceptable composition of the present invention, include those broadly defined as compounds produced by the condensation of alkylene oxide groups (hydrophilic in nature) with an organic hydrophobic compound, which may be aliphatic or alkyl aromatic in nature. Examples of preferred classes of nonionic surface-active agents are: the long chain alkanolamides; the polyethylene oxide condensates of alkyl phenols; the condensation product of aliphatic alcohols having from about 8 to about 18 carbon atoms, in either straight chain or branched chain configuration, with ethylene oxide; the long chain tertiary amine oxides; the long chain tertiary phosphine oxides; the long chain dialkyl sulfoxides containing one short chain alkyl or hydroxy alkyl radical of from about 1 to about 3 carbon atoms; and the alkyl polysaccharide (APS) surfactants such as the alkyl polyglycosides; the polyethylene glycol (PEG) glyceryl fatty esters.

Zwitterionic surface-active agents such as betaines can also be useful in the cosmetically acceptable composition of this invention. Examples of betaines useful herein include the high alkyl betaines, such as coco dimethyl carboxymethyl betaine, cocoamidopropyl betaine, cocobetaine, lauryl amidopropyl betaine, oleyl betaine, lauryl dimethyl carboxymethyl betaine, lauryl dimethyl alphacarboxyethyl betaine, cetyl dimethyl carboxymethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, and lauryl bis-(2-hydroxypropyl)alpha-carboxyethyl betaine. The sulfobetaines may be represented by coco dimethyl sulfopropyl betaine, stearyl dimethyl sulfopropyl betaine, lauryl dimethyl sulfoethyl betaine, lauryl bis-(2-hydroxyethyl) sulfopropyl betaine and the like; amidobetaines and amidosulfobetaines, wherein the RCONH(CH.sub.2).sub.3 radical is attached to the nitrogen atom of the betaine are also useful in this invention.

The anionic, cationic, nonionic, amphoteric or zwitterionic surface-active agents used in the cosmetically acceptable composition of the present invention are typically used in an amount from about 0.1 to 50 percent by weight, preferably from about 0.5 to about 40 percent by weight, more preferably from about 1 to about 20 percent by weight.

The cosmetically acceptable compositions of the present invention may include humectants, which act as hygroscopic agents, increasing the amount of water absorbed, held and retained. Suitable humectants for the formulations of this invention include but are not limited to: acetamide MEA, ammonium lactate, chitosan and its derivatives, colloidal oatmeal, galactoarabinan, glucose glutamate, glerecyth-7, glygeryth-12, glycereth-26, glyceryth-31, glycerin, lactamide MEA, lactamide DEA, lactic acid, methyl gluceth-10, methyl gluceth-20, panthenol, propylene glycol, sorbitol, polyethylene glycol, 1,3-butanediol, 1,2,6-hexanetriol, hydrogenated starch hydrolysate, inositol, mannitol, PEG-5 pentaerythritol ether, polyglyceryl sorbitol, xylitol, sucrose, sodium hyaluronate, sodium PCA, and combinations thereof. Glycerin is a particularly preferred humectant. The humectant is present in the composition at concentrations of from about 0.5 to about 40 percent by weight, preferably from about 0.5 to about 20 percent by weight and more preferably from about 0.5 to about 12 percent by weight.

The cosmetically acceptable compositions of the present invention may include petrolatum or mineral oil components, which when selected will generally be USP or NF grade. The petrolatum may be white or yellow. The viscosity or consistency grade of petrolatum is not narrowly critical. Petrolatum can be partially replaced with mixtures of hydrocarbon materials, which can be formulated to resemble petrolatum in appearance and consistency. For example, mixtures of petrolatum or mineral oil with different waxes and the like may be combined. Preferred waxes include bayberry wax, candelilla wax, ceresin, jojoba butter, lanolin wax, montan wax, ozokerite, polyglyceryl-3-beeswax, polyglyceryl-6-pentastearate, microcrystalline wax, paraffin wax, isoparaffin, vaseline solid paraffin, squalene, oligomer olefins, beeswax, synthetic candelilla wax, synthetic carnauba, synthetic beeswax and the like may be blended together. Alkylmethyl siloxanes with varying degrees of substitution can be used to increase water retained by the skin. Siloxanes such as stearyl dimethicone, known as 2503 Wax, C30-45 alkyl methicone, known as AMS-C30 wax, and stearoxytrimethylsilane (and) stearyl alcohol, known as 580 Wax, each available from Dow Coming, Midland, Mich., USA. Additional alkyl and phenyl silicones may be employed to enhance moisturizing properties. Resins such as dimethicone (and) trimethylsiloxysilicate or Cyclomethicone (and) Trimethylsiloxysilicate fluid, may be utilized to enhance film formation of skin care products. When used, the petrolatum, wax or hydrocarbon or oil component is included in the formulations at a concentration of about 1 to about 20 weight percent, more preferably about 1 to about 12 weight percent. When used, the silicone resins can be included from about 0.1 to about 10.0 weight percent.

Emollients are defined as agents that help maintain the soft, smooth, and pliable appearance of skin. Emollients function by their ability to remain on the skin surface or in the stratum corneum. The cosmetically acceptable composition of the present invention may include fatty ester emollients, which are listed in the International Cosmetic Ingredient Dictionary, Eighth Edition, 2000, p. 1768 to 1773. Specific examples of suitable fatty esters for use in the formulation of this invention include isopropyl myristate, isopropyl palmitate, caprylic/capric triglycerides, cetyl lactate, cetyl palmitate, hydrogenated castor oil, glyceryl esters, hydroxycetyl isostearate, hydroxy cetyl phosphate, isopropyl isostearate, isostearyl isostearate, diisopropyl sebacate, PPG-5-Ceteth-20, 2-ethylhexyl isononoate, 2-ethylhexyl stearate, C.sub.12 to C.sub.16 fatty alcohol lactate, isopropyl lanolate, 2-ethyl-hexyl salicylate, and mixtures thereof. The presently preferred fatty esters are isopropyl myristate, isopropyl palmitate, PPG-5-Ceteth-20, and caprylic/capric triglycerides. When used the fatty ester emollient is preferably included in the formulations of this invention at a concentration of about 1 to about 8 weight percent, more preferably about 2 to about 5 weight percent.

The compositions of the present invention may also include silicone compounds. Preferably, the viscosity of the silicone component is from about 0.5 to about 12,500 cps. Examples of suitable materials are dimethylpolysiloxane, diethylpolysiloxane, dimethylpolysiloxane-diphenylpolysiloxane, cyclomethicone, trimethylpolysiloxane, diphenylpolysiloxane, and mixtures thereof. Dimethicone, a dimethylpolysiloxane endblocked with trimethyl units, is one preferred example. Dimethicone having a viscosity between 50 and 1,000 cps is particularly preferred. When used, the silicone oils are preferably included in the formulations of this invention at a concentration of 0.1 to 5 weight percent, more preferably 1 to 2 weight percent.

The cosmetically acceptable compositions of the present invention may include volatile and non-volatile silicone oils or fluids. The silicone compounds can be either linear or cyclic polydimethylsiloxanes with a viscosity from about 0.5 to about 100 centistokes. The most preferred linear polydimethylsiloxane compounds have a range from about 0.5 to about 50 centistokes. One example of a linear, low molecular weight, volatile polydimethylsiloxane is octamethyltrisiloxane. 200 fluid having a viscosity of about 1 centistoke. When used, the silicone oils are preferably included in the formulations of this invention at a concentration of 0.1 to 30 weight percent, more preferably 1 to 20 weight percent.

The cosmetically acceptable compositions of the present invention may include volatile, cyclic, low molecular weight polydimethylsiloxanes (cyclomethicones). The preferred cyclic volatile siloxanes can be polydimethyl cyclosiloxanes having an average repeat unit of 4 to 6, and a viscosity from about 2.0 to about 7.0 centistokes, and mixtures thereof. Preferred cyclomethicones are available from Dow Corning, Midland, Mich., and from General Electric, Waterford, N.Y., USA. When used, the silicone oils are preferably included in the formulations of this invention at a concentration of 0.1 to 30 weight percent, more preferably 1 to 20 weight percent.

Silicone surfactants or emulsifiers with polyoxyethylene or polyoxypropylene side chains may also be used in compositions of the present invention. Preferred examples include dimethicone copolyols and 5225C Formulation Aids, available from Dow Coming, Midland, Mich., USA and Silicone SF-1528, available from General Electric, Waterford, N.Y., USA. The side chains may also include alkyl groups such as lauryl or cetyl. Preferred are lauryl methicone copolyol. 5200 ormulation Aid, and cetyl dimethicone copolyol, known as Abil EM-90, available from Goldschmidt Chemical Corporation, Hopewell, Va. Also preferred is lauryl dimethicone, known as Belsil LDM 3107 VP, available from Wacker-Chemie, Munchen, Germany. When used, the silicone surfactants are preferably included in the formulations of this invention at a concentration of 0.1 to 30 weight percent, more preferably 1 to 15 weight percent. Amine functional silicones and emulsions may be utilized in the present invention. Preferred examples include Dow Coming 8220, Dow Coming 939, Dow Coming 949, Dow Coming 2-8194, all available from Dow Coming, Midland, Mich., USA. Also preferred is Silicone SM 253 available from General Electric, Waterford, N.Y., USA. When used, the amine functional silicones are preferably included in the formulations of this invention at a concentration of 0.1 to 5 weight percent, more preferably 0.1 to 2.0 weight percent.

The cosmetically acceptable compositions of the present invention may include volatile hydrocarbon oils. The volatile hydrocarbon comprises from about C.sub.6 to C.sub.22 atoms. A preferred volatile hydrocarbon is an aliphatic hydrocarbon having a chain length from about C.sub.6 to C.sub.16 carbon atoms. An example of such compound includes isohexadecane, under the tradename Permethyl 101A, available from Presperse, South Plainfield, N.J., USA. Another example of a preferred volatile hydrocarbon is C.sub.12 to C.sub.14 isoparaffin, under the tradename Isopar M, available from Exxon, Baytown, Tex., USA. When used, the volatile hydrocarbons are preferably included in the formulations of this invention at a concentration of 0.1 to 30 weight percent, more preferably 1 to 20 weight percent.

The cosmetically acceptable compositions of the present invention may include cationic and ampholytic conditioning polymers. Examples of such include, but are not limited to those listed by the International Cosmetic Ingredient Dictionary published by the Cosmetic, Toiletry, and Fragrance Association (CTFA), 1101 17 Street, N.W., Suite 300, Washington, D.C. 20036. General examples include quaternary derivatives of cellulose ethers, quaternary derivatives of guar, homopolymers and copolymers of DADMAC, homopolymers and copolymers of MAPTAC and quaternary derivatives of starches. Specific examples, using the CTFA designation, include, but are not limited to Polyquaternium-10, Guar hydroxypropyltrimonium chloride, Starch hydroxypropyltrimonium chloride, Polyquaternium-4, Polyquaternium-5, Polyquaternium-6, Polyquaternium-7, Polyquaternium-14, Polyquaternium-15, Polyquaternium-22, Polyquaternium-24, Polyquaternium-28, Polyquaternium-32, Polyquaternium-33, Polyquaternium-36, Polyquaternium-37, Polyquaternium-39, Polyquaternium-45, Polyquaternium-47 and polymethacrylamidopropyltrimonium chloride, and mixtures thereof. When used, the conditioning polymers are preferably included in the cosmetically acceptable composition of this invention at a concentration of from 0.1 to 10 weight percent, preferably from 0.2 to 6 weight percent and most preferably from 0.2 to 5 weight percent.

The cosmetically acceptable compositions of the present invention may include one or more rheological modifiers. The rheological modifiers that can be used in this invention include, but are not limited to high molecular weight crosslinked homopolymers of acrylic acid, and Acrylates/C10-30 Alkyl Acrylate Crosspolymer, such as the Carbopol. and Pemulen series, both available from B. F. Goodrich, Akron, Ohio, USA; anionic acrylate polymers such as Salcare and cationic acrylate polymers such as Salcare SC96, available from Ciba Specialties, High Point, N.C., USA; Acrylamidopropylttrimonium chloride/acrylamide; Hydroxyethyl methacrylates polymers, Steareth-10 Allyl Ether/Acrylate Copolymer; Acrylates/Beheneth-25 Metacrylate Copolymer, known as Aculyn, available from International Specialties, Wayne, N.J., USA; Glyceryl Polymethacrylate, Acrylates/Steareth-20 Methacrylate Copolymer; bentonite; gums such as alginates, carageenans, gum acacia, gum arabic, gum ghatti, gum karaya, gum tragacanth, guar gum; guar hydroxypropyltrimonium chloride, xanthan gum or gellan gum; cellulose derivatives such as sodium carboxymethyl cellulose, hydroxyethyl cellulose, hydroxymethyl carboxyethyl cellulose, hydroxymethyl carboxypropyl cellulose, ethyl cellulose, sulfated cellulose, hydroxypropyl cellulose, methyl cellulose, hydroxypropylmethyl cellulose, microcrystalline cellulose; agar; pectin; gelatin; starch and its derivatives; chitosan and its derivatives such as hydroxyethyl chitosan; polyvinyl alcohol, PVM/MA copolymer, PVM/MA decadiene crosspolymer, poly(ethylene oxide) based thickeners, sodium carbomer, and mixtures thereof. When used, the rheology modifiers are preferably included in the cosmetically acceptable composition of this invention at a concentration of from 0.01 to 12 weight percent, preferably from 0.05 to 10 weight percent and most preferably from 0.1 to 6 weight percent.

The cosmetically acceptable composition of the present invention may include one or more antioxidants, which include, but are not limited to ascorbic acid, BHT, BHA, erythorbic acid, bisulfite, thioglycolate, tocopherol, sodium metabisulfite, vitamin E acetate, and ascorbyl palmitate. The antioxidants will be present at from 0.01 to 5 weight percent, preferably 0.1 to 3 weight percent and most preferably from 0.2 to 2 weight percent of the cosmetically acceptable composition.

The cosmetically acceptable compositions of the present invention may include one or more sunscreen active agents. Examples of sunscreen active agents include, but are not limited to octyl methoxycinnamate (ethylhexyl p-methoxycinnamate), octyl salicylate oxybenzone (benzophenone-3), benzophenone-4, menthyl anthranilate, dioxybenzone, aminobenzoic acid, amyl dimethyl PABA, diethanolamine p-methoxy cinnamate, ethyl 4-bis (hydroxypropyl) aminobenzoate, 2-ethylhexy 1-2-cyano-3, 3-diphenylacrylate, homomenthyl salicylate, glyceryl aminobenzoate, dihydroxyacetone, octyl dimethyl PABA, 2-phenylbenzimidazole-5-sulfonic acid, triethanolamine salicylate, zinc oxide, and titanium oxide, and mixtures thereof. The amount of sunscreen used in the cosmetically acceptable composition of this invention will vary depending on the specific UV absorption wavelength(s) of the specific sunscreen active(s) used and can be from 0.1 to 10 percent by weight, from 2 to 8 percent by weight.

The cosmetically acceptable compositions of the present invention may include one or more preservatives. Example of preservatives, which may be used include, but are not limited to 1,2-dibromo-2,4-dicyano butane (Methyldibromo Glutaronitrile, known as MERGUARD. Nalco Chemical Company, Naperville, Ill., USA), benzyl alcohol, imidazolidinyl urea, 1,3-bis (hydroxymethyl)-5,5-dimethyl-2,3-imidazolidinedione (e.g., DMDM Hydantoin, known as GLYDANT, Lonza, Fairlawn, N.J., USA.), methylchloroisothiazolinone and methylisothiazolinone (e.g., Kathon, Rohm & Haas Co., Philadelphia, Pa., USA), methyl paraben, propyl paraben, phenoxyethanol, and sodium benzoate, and mixtures thereof.

The cosmetically acceptable compositions of the present invention may include any other ingredient by normally used in cosmetics. Examples of such ingredients include, but are not limited to buffering agents, fragrance ingredients, chelating agents, color additives or dyestuffs which can serve to color the composition itself or keratin, sequestering agents, softeners, foam synergistic agents, foam stabilizers, sun filters and peptizing agents.

The surface of pigments, such titanium dioxide, zinc oxide, talc, calcium carbonate or kaolin, can be treated with the unsaturated quaternary ammonium compounds described herein and then used in the cosmetically acceptable composition of the present invention. The treated pigments are then more effective as sunscreen actives and for use in color cosmetics such as make up and mascara.

The cosmetically acceptable compositions of the present invention can be presented in various forms. Examples of such forms include, but are not limited a solution, liquid, cream, emulsion, dispersion, gel, thickening lotion.

The cosmetically acceptable compositions of the present invention may contain water and also any cosmetically acceptable solvent. Examples of acceptable solvents include, but are not limited to monoalcohols, such as alkanols having 1 to 8 carbon atoms (like ethanol, isopropanol, benzyl alcohol and phenylethyl alcohol) polyalcohols, such as alkylene glycols (like glycerine, ethylene glycol and propylene glycol) and glycol ethers, such as mono-, di- and tri-ethylene glycol monoalkyl ethers, for example ethylene glycol monomethyl ether and diethylene glycol monomethyl ether, used singly or in a mixture. These solvents can be present in proportions of up to as much as 70 percent by weight, for example from 0.1 to 70 percent by weight, relative to the weight of the total composition.

The cosmetically acceptable compositions of the present invention can also be packaged as an aerosol, in which case it can be applied either in the form of an aerosol spray or in the form of an aerosol foam. As the propellant gas for these aerosols, it is possible to use, in particular, dimethyl ether, carbon dioxide, nitrogen, nitrous oxide, air and volatile hydrocarbons, such as butane, isobutane, and propane.

The cosmetically acceptable compositions of the present invention can contain electrolytes, such as aluminum chlorohydrate, alkali metal salts, e.g., sodium, potassium or lithium salts, these salts preferably being halides, such as the chloride or bromide, and the sulfate, or salts with organic acids, such as the acetates or lactates, and also alkaline earth metal salts, preferably the carbonates, silicates, nitrates, acetates, gluconates, pantothenates and lactates of calcium, magnesium and strontium.

Compositions of the present invention can include leave-on or rinse-off skin care products such as lotions, hand/body creams, shaving gels or shaving creams, body washes, sunscreens, liquid soaps, deodorants, antiperspirants, suntan lotions, after sun gels, bubble baths, hand or mechanical dishwashing compositions, and the like. In addition to the polymer, skin care compositions may include components conventionally used in skin care formulations. Such components include for example; (a) humectants, (b) petrolatum or mineral oil, (c) fatty alcohols, (d) fatty ester emollients, (e) silicone oils or fluids, and (f) preservatives. These components must in general be safe for application to the human skin and must be compatible with the other components of the formulation. Selection of these components is generally within the skill of the art. The skin care compositions may also contain other conventional additives employed in cosmetic skin care formulations. Such additives include aesthetic enhancers, fragrance oils, dyes and medicaments such as menthol and the like.

The skin care compositions of the present invention may be prepared as oil-in-water, water-in-oil emulsions, triple emulsions, or dispersions.

Preferred oil-in-water emulsions are prepared by first forming an aqueous mixture of the water-soluble components, e.g. unsaturated quaternary ammonium compounds, humectants, water-soluble preservatives, followed by adding water-insoluble components. The water-insoluble components include the emulsifier, water-insoluble preservatives, petrolatum or mineral oil component, fatty alcohol component, fatty ester emollient, and silicone oil component. The input of mixing energy will be high and will be maintained for a time sufficient to form a water-in-oil emulsion having a smooth appearance (indicating the presence of relatively small micelles in the emulsion). Preferred dispersions are generally prepared by forming an aqueous mixture of the water-soluble components, followed by addition of thickener with suspension power for water-insoluble materials.

Compositions of the present invention for treating hair include bath preparations such as bubble baths, soaps, and oils, shampoos, conditioners, hair bleaches, hair coloring preparations, temporary and permanent hair colors, color conditioners, hair lighteners, coloring and non-coloring hair rinses, hair tints, hair wave sets, permanent waves, curling, hair straighteners, hair grooming aids, hair tonics, hair dressings and oxidative products. The dispersion polymers may also be utilized in styling type leave-in products such as gels, mousses, spritzes, styling creams, styling waxes, pomades, balms, and the like, either alone or in combination with other polymers or structuring agents in order to provide control and hair manageability with a clean, natural, non-sticky feel.

Hair care compositions of the present invention give slippery feel and that can be easily rinsed from the hair due to the presence of the dispersion polymer, volatile silicones, other polymers, surfactants or other compounds that may alter the deposition of materials upon the hair.

In the case of cleansing formulations such as a shampoo for washing the hair, or a liquid hand soap, or shower gel for washing the skin, the compositions of the present invention contain anionic, cationic, nonionic, zwitterionic or amphoteric surface-active agents typically in an amount from about 3 to about 50 percent by weight, preferably from about 3 to about 20 percent, and their pH is general in the range from about 3 to about 10.

Preferred shampoos of the present invention contain combinations of anionic surfactants with zwitterionic surfactants and/or amphoteric surfactants. Especially preferred shampoos contain from about 0 to about 16 percent active of alkyl sulfates, from 0 to about 50 weight percent of ethoxylated alkyl sulfates, and from 0 to about 50 weight percent of optional surface-active agents selected from the nonionic, amphoteric, and zwitterionic surface-active agents, with at least 5 weight percent of either alkyl sulfate, ethoxylated alkyl sulfate, or a mixture thereof, and a total surfactant level of from about 10 weight to about 25 percent.

The shampoo for washing hair also can contain other conditioning additives such as silicones and conditioning polymers typically used in shampoos. U.S. Pat. No. 5,573,709 provides a list of non-volatile silicone conditioning agents that can be used in shampoos. The conditioning polymers for use with the present invention are listed in the Cosmetic, Toiletries and Fragrance Associations (CTFA) dictionary. Specific examples include the Polyquaterniums (example Polyquaternium-1 to Polyquaternium-50), Guar Hydroxypropyl Trimonium Chloride, Starch Hydroxypropyl Trimonium Chloride and Polymethacrylamidopropyl Trimonium Chloride.

Other preferred embodiments consist of use in the form of a rinsing lotion to be applied mainly before or after shampooing. These lotions typically are aqueous or aqueous-alcoholic solutions, emulsions, thickened lotions or gels. If the compositions are presented in the form of an emulsion, they can be nonionic, anionic or cationic. The nonionic emulsions consist mainly of a mixture of oil and/or a fatty alcohol with a polyoxyethyleneated alcohol, such as polyoxyethyleneated stearyl or cetyl/stearyl alcohol, and cationic surface-active agents can be added to these compositions. The anionic emulsions are formed essentially from soap.

If the compositions of the present invention are presented in the form of a thickened lotion or a gel, they contain thickeners in the presence or absence of a solvent. The thickeners which can be used are especially resins, Carbopol-type acrylic acid thickeners available from B.F. Goodrich; xanthan gums; sodium alginates; gum arabic; cellulose derivatives and poly-(ethylene oxide) based thickeners, and it is also possible to achieve thickening by means of a mixture of polyethylene glycol stearate or distearate or by means of a mixture of a phosphoric acid ester and an amide. The concentration of thickener is generally 0.05 to 15 percent by weight. If the compositions are presented in the form of a styling lotion, shaping lotion, or setting lotion, they generally comprise, in aqueous, alcoholic or aqueous-alcoholic solution, the ampholyte polymers defined above.

In the case of hair fixatives, the composition of the present invention may also contain one or more additional hair fixative polymers. When present, the additional hair fixative polymers are present in a total amount of from about 0.25 to about 10 percent by weight. The additional hair fixative resin can be selected from the following group as long as it is compatible with a given dispersion polymer: acrylamide copolymer, acrylamide/sodium acrylate copolymer, acrylate/ammonium methacrylate copolymer, an acrylate copolymer, an acrylic/acrylate copolymer, adipic acid/dimethylaminohydroxypropyl diethylenetriamine copolymer, adipic acid/epoxypropyl diethylenetriamine copolymer, allyl stearate/VA copolymer, aminoethylacrylate phosphate/acrylate copolymer, an ammonium acrylate copolymer, an ammonium vinyl acetate/acrylate copolymer, an AMP acrylate/diacetoneacrylamide copolymer, an AMPD acrylate/diacetoneacrylamide copolymer, butyl ester of ethylene/maleic anhydride copolymer, butyl ester of PVM/MA copolymer, calcium/sodium PVM/MA copolymer, corn starch/acrylamide/sodium acrylate copolymer, diethylene glycolamine/epichlorohydrin/piperazine-copolymer, dodecanedioic acid/cetearyl alcohol/glycol copolymer, ethyl ester of PVM/MA copolymer, isopropyl ester of PVM/MA copolymer, karaya gum, a methacryloyl ethyl betaine/methacrylate copolymer, an octylacrylamide/acrylate/butylaminoethyl methacrylate copolymer, an octylacrylamide/acrylate copolymer, phthalic anhydride/glycerin/glycidyl decanoate copolymer, a phthalic/trimellitic/glycol copolymer, polyacrylamide, polyacrylamidomethylpropane sulfonic acid, polybutylene terephthalate, polyethylacrylate, polyethylene, polyquaternium-1, polyquaternium-2, polyquaternium-4, polyquaternium-5, polyquaternium-6, polyquaternium-7, polyquaternium-8, polyquaternium-9, polyquaternium-10, polyquaternium-11, polyquaternium-12, polyquaternium-13, polyquaternium-14, polyquaternium-15, polyquaternium-39, polyquaternium-47, polyvinyl acetate, polyvinyl butyral, polyvinyl imidazolinium acetate, polyvinyl methyl ether, PVM/MA copolymer, PVP, PVP/dimethylaminoethylmethacrylate copolymer, PVP/eicosene copolymer, PVP/ethyl methacrylate/methacrylic acid copolymer, PVP/hexadecene copolymer, PVP/VA copolymer, PVP/vinyl acetate/itaconic acid copolymer, shellac, sodium acrylates copolymer, sodium acrylates/Acrylnitrogens copolymer, sodium acrylate/vinyl alcohol copolymer, sodium carrageenan, starch diethylaminoethyl ether, stearylvinyl ether/maleic anhydride copolymer, sucrose benzoate/sucrose acetate isobutyrate/butyl benzyl phthalate copolymer, sucrose benzoate/sucrose acetate isobutyrate/butyl benzyl phthalate/methyl methacrylate copolymer, sucrose benzoate/sucrose acetate isobutyrate copolymer, a vinyl acetate/crotonate copolymer, vinyl acetate/crotonic acid copolymer, vinyl acetate/crotonic acid/methacryloxybenzophenone-1 copolymer, vinyl acetate/crotonic acid/vinyl neodecanoate copolymer, and mixtures thereof. Synthetic polymers used for creating styling aids are described in “The History of Polymers in Haircare,” Cosmetics and Toiletries, 103 (1988), incorporated herein by reference. Other synthetic polymers that may be used with the present invention can be referenced in the CTFA Dictionary, Fifth Edition, 2000, incorporated herein by reference.

The cosmetically acceptable carrier contained in the cosmetic compositions of the present invention may be varied depending on the type of the formulation. For example, the formulation of ointment, pastes, creams or gels may comprise animal and vegetable fats, waxes, paraffins, starch, tragacanth, cellulose derivatives, polyethylene glycols, silicones, bentonites, silica, talc, zinc oxide or mixtures of these ingredients.

In the formulation of powder or spray, it may comprise lactose, talc, silica, aluminum hydroxide, calcium silicate, polyamide powder and mixtures of these ingredients. Spray may additionally comprise the customary propellants, for example, chlorofluorohydrocarbons, propane, butane, diethyl ether, or dimethyl ether.

The formulation of solution and emulsion of the present invention may comprise solvent, solubilizer and emulsifier, for example water, ethanol, isopropanol, ethyl carbonate, ethyl acetate, benzyl alcohol, benzyl benzoate, propylene glycol, 1,3-butyleneglycol, oils, in particular cottonseed oil, groundnut oil, maize germ oil, olive oil, castor oil and sesame seed oil, glycerol fatty esters, polyethylene glycol and fatty acid esters of sorbitan or mixtures of these ingredients.

The formulation of suspension of the present invention may comprise liquid diluents, for example water, ethanol or propylene glycol, suspending agents, for example ethoxylated isosteary alcohols, polyoxyethylene sorbitol esters and poly oxyethylene sorbitan esters, micocrystalline cellulose, aluminum metahydroxide, bentonite, agar and tragacanth or mixtures of these ingredients.

The formulation of cleansing compositions of the present invention with surfactant may comprise aliphatic alcohol sulfate, aliphatic alcohol ether sulfate, sulfosucinnate monoester, isothinate, imidazolium derivatives, methyltaurate, sarcocinate, fatty acid amide ether sulfate, alkyl amido betain, aliphatic alcohol, fatty acid glyceride, fatty acid diethanolamide, vegetable oil, lanoline derivatives, ethoxylated glycerol fatty acid ester or mixtures of these ingredients.

Additional antioxidant ingredients and compositions can be selected from, but not limited to, Ascorbic acid, Ascorbic acid derivatives, Glucosamine ascorbate, Arginine ascorbate, Lysine ascorbate, Glutathione ascorbate, Nicotinamide ascorbate, Niacin ascorbate, Allantoin ascorbate, Creatine ascorbate, Creatinine ascorbate, Chondroitin ascorbate, Chitosan ascorbate, DNA Ascorbate, Carnosine ascorbate, Vitamin E, various Vitamin E derivatives, Tocotrienol, Rutin, Quercetin, Hesperedin (Citrus sinensis), Diosmin (Citrus sinensis), Mangiferin (Mangifera indica), Mangostin (Garcinia mangostana), Cyanidin (Vaccinium myrtillus), Astaxanthin (Haematococcus algae), Lutein (Tagetes patula), Lycopene (Lycopersicum esculentum), Resveratrol (Polygonum cuspidatum), Tetrahydrocurcumin (Curcuma longa), Rosmarinic acid (Rosmarinus officinalis), Hypericin (Hypericum perforatum), Ellagic acid (Punica granatum), Chlorogenic acid (Vaccinium vulgaris), Oleuropein (Olea europaea), a-Lipoic acid, Niacinamide lipoate, Glutathione, Andrographolide (Andrographis paniculata), Carnosine, Niacinamide, Potentilla erecta extract, Polyphenols, Grapeseed extract, Pycnogenol (Pine Bark extract), Pyridoxine, Magnolol, Honokiol, Paeonol, Resacetophenone, Quinacetophenone, arbutin, kojic acid, and combinations thereof.

The blood micro-circulation improvement ingredients and compositions can be added to compositions of the present invention. These are selected from Horse Chestnut Extract (Aesculus hippocastanum extract)), Esculin, Escin, Yohimbine, Capsicum Oleoresin, Capsaicin, Niacin, Niacin Esters, Methyl Nicotinate, Benzyl Nicotinate, Ruscogenins (Butchers Broom extract; Ruscus aculeatus extract), Diosgenin (Trigonella foenumgraecum, Fenugreek), Emblica extract (Phyllanthus emblica extract), Asiaticoside (Centella asiatica extract), Boswellia Extract (Boswellia serrata), Ginger Root Extract (Zingiber Officianalis), Piperine, Vitamin K, Melilot (Melilotus officinalis extract), Glycyrrhetinic acid, Ursolic acid, Sericoside (Terminalia sericea extract), Darutoside (Siegesbeckia orientalis extract), Amni visnaga extract, extract of Red Vine (Vitis Vinifera) leaves, apigenin, phytosan, luteolin, and combinations thereof.

The anti-inflammatory ingredients can be added to compositions of the present invention. These can be selected from at least one antioxidant class of Cyclo-oxygenase (for example, COX-1 or COX-2) or Lipoxygenase (for example, LOX-5) enzyme inhibitors such as Ascorbic acid, Ascorbic acid derivatives, Vitamin E, Vitamin E derivatives, Tocotrienol, Rutin, Quercetin, Hesperedin (Citrus sinensis), Diosmin (Citrus sinensis), Mangiferin (Mangifera indica), Mangostin (Garcinia mangostana), Cyanidin (Vaccinium myrtillus), Astaxanthin (Haematococcus algae), Lutein (Tagetes patula), Lycopene (Lycopersicum esculentum), Resveratrol (Polygonum cuspidatum), Tetrahydrocurcumin (Curcuma longa), Rosmarinic acid (Rosmarinus officinalis), Hypericin (Hypericum perforatum), Ellagic acid (Punica granatum), Chlorogenic acid (Vaccinium vulgaris), Oleuropein (Olea europaea), alpha-Lipoic acid, Glutathione, Andrographolide, Grapeseed extract, Green Tea Extract, Polyphenols, Pycnogenol (Pine Bark extract), White Tea extract, Black Tea extract, (Andrographis paniculata), Carnosine, Niacinamide, and Emblica extract. Anti-inflammatory composition can additionally be selected from, but not limited to, Horse Chestnut Extract (Aesculus hippocastanum extract)), Esculin, Escin, Yohimbine, Capsicum Oleoresin, Capsaicin, Niacin, Niacin Esters, Methyl Nicotinate, Benzyl Nicotinate, Ruscogenins (Butchers Broom extract; Ruscus aculeatus extract), Diosgenin (Trigonella foenumgraecum, Fenugreek), Emblica extract (Phyllanthus emblica extract), Asiaticoside (Centella asiatica extract), Boswellia Extract (Boswellia serrata), Sericoside, Visnadine, Thiocolchicoside, Grapeseed Extract, Ginger Root Extract (Zingiber Officianalis), Piperine, Vitamin K, Melilot (Melilotus officinalis extract), Glycyrrhetinic acid, Ursolic acid, Sericoside (Terminalia sericea extract), Darutoside (Siegesbeckia orientalis extract), Amni visnaga extract, extract of Red Vine (Vitis-Vinifera) leaves, apigenin, phytosan, luteolin, and combinations thereof.

Certain divalent metal ions can be added to compositions of the present invention. The examples of such metal ions include zinc, copper, manganese, vanadium, chromium, cobalt, selenium, molybdenum, and iron.

### EXAMPLES

The following examples are presented to illustrate presently preferred practice thereof. These examples also include the formulation of consumer desirable lotion, cream, and other such compositions for their retail marketing. As illustrations they are not intended to limit the scope of the invention. All quantities are in weight percent.

### Example 1

Lignan Hair Loss Prevention Serum.

Ingredients. (1) Deionized water 20.0 (2) Verbascoside (10 percent solution) 5.0 (3) Methylpropanediol 69.5 (4) Dimethicone copolyol 4.0 (5) Preservatives 0.5 (6) Ammonium Acryloyldimethyltaurate/VP copolymer 1.0. Procedure. Make main batch by mixing (2) to (5) at room temperature. Pre-mix (1) and (6) to a clear paste and add to main batch with mixing. The product has a clear to slightly hazy syrup-like appearance, typical of a skin serum product. Upon application to hair by the method of the present invention it is absorbed rapidly with a silky smooth skin feel on scalp giving shiny appearance to hair.

### Example 2

Lignan Skin Brightening Serum.

Ingredients. (1) Deionized water 20.0 (2) Rosmarinic Acid 5.0 (3) Methylpropanediol 69.0 (4) Dimethicone copolyol 4.0 (5) Preservatives 0.5 (6) Protodioscin 0.5. (7) Ammonium Acryloyldimethyltaurate/VP copolymer 1.0 Procedure. Make main batch by mixing (2) to (6) at room temperature. Pre-mix (1) and (7) to a clear paste and add to main batch with mixing. The product has a clear to slightly hazy syrup-like appearance, typical of a skin serum product. Upon application to skin by the method of the present invention it is absorbed rapidly with a silky smooth skin feel.

### Example 3

Lignan Wrinkle Reduction Cream.

Ingredients. (1) Deionized water 79.5 (2) Cetearyl alcohol (and) dicetyl phosphate (and) Ceteth-10 phosphate 5.0 (3) Cetyl alcohol 2.0 (4) Glyceryl stearate (and) PEG-100 stearate 4.0 (5) Triethyl citrate 5.0 (6) Secoisolariciresinol 3.0 (7) Hecogenin 1.0 (8) (8) Preservatives 0.5. Procedure. Mix 1 to 5 and heat to 75-80° C. Adjust pH to 4.0 4.5. Cool to 35-40 C with mixing. Add 6 to 8 with mixing. Adjust pH to 4.0-4.5, if necessary. White to off-white cream.

### Example 4

Lignan Eyelid Puffiness Reduction Facial Mask.

Ingredients. (1) Chitosan 5.0 (2) Verbascoside 4.2 (3) PEG-6 17.7 (4) Water 70.6 (5) Carnosine 0.5 (6) Zinc Salicylate 0.5 (7) Silybin 1.0 (8) Preservatives 0.5. Procedure: Mix 1 and 4 to a clear gel-like paste. Mix all other ingredients separately and add this to main batch and mix. A clear gel product is obtained. It is applied on the face, under the eye-lids, and the neck and left for 10 to 30 minutes, then rinsed off.

### Example 5

Lignan Wound Healing Cream.

Ingredients. (1) Water 65.4 (2) Dicetyl Phosphate (and) Ceteth-10 Phosphate 5.0 (3) Glyceryl Stearate (and) PEG-100 Stearate 4.0 (4) Phenoxyethanol 0.7 (5) Chlorphenesin 0.3 (60) Titanium Dioxide 0.2 (7) Sodium Hydroxide 0.5 (8) Magnolol 0.2 (9) Boswellia Serrata 0.5 (10) Cetyl Dimethicone 1.5 (11) Silybin 0.5 (12) Shea butter 2.0 (13) Rosmarinic Acid 1.0 (14) Water 5.0 (15) Zinc Lactate 1.0 (16) Polyamide-3 3.0 (17) 2,4-Dihydroxy Acetophenone (Resacetophenone) 1.1 (18) Triethyl citrate 1.5 (19) Carnosine 0.1 (20) Cyclomethicone, Dimethicone Crosspolymer 2.0 (21) Arbutin 0.5 (22) Polysorbate-20 2.0 (23) Sepigel-305 2.0. Procedure. Mix (1) to (22) and heat at 70 to 80 C till homogenous. Cool to 40 to 50 C. Cool to room temperature, then add (23) to a desired viscosity. An off-white cream is obtained.

### Example 6

Lignan Acne Cream with Sebum Reduction.

Ingredients. (1) Water 61.0 (2) Dicetyl Phosphate (and) Ceteth-10 Phosphate 5.0 (3) Glyceryl Stearate (and) PEG-100 Stearate 4.0 (4) Phenoxyethanol 0.7 (5) Chlorphenesin 0.3 (60) Titanium Dioxide 0.2 (7) Sodium Hydroxide 0.5 (8) Magnolol 0.2 (9) Boswellia Serrata 0.5 (10) Cetyl Dimethicone 1.5 (11) Tetrahydrocurcuminoids 0.5 (12) Shea butter 2.0 (13) Ximenia oil 1.0 (14) Water 5.0 (15) Dioscin 4.0 (16) Sesamin 2.2 (17) 2,4-Dihydroxy Acetophenone (Resacetophenone) 1.1 (18) Triethyl citrate 2.2 (19) Carnosine 0.1 (20) Cyclomethicone, Dimethicone Crosspolymer 2.0 (21) Arbutin 0.5 (22) Zinc Salicylate 1.5 (23) Polysorbate-20 2.0 (24) Sepigel-305 2.0. Procedure. Mix (1) to (23) and heat at 70 to 80 C till homogenous. Cool to room temperature, and add (24) to a desired viscosity. An off-white cream is obtained.

### Example 7

Acne Cleanser with Facial Oil Reduction.

Ingredients. (1) PEG-6 57.12 (2) Hydroxypropyl Cellulose 0.3 (3) Boswellia Serrata 0.05 (4) Sodium Cocoyl Isethionate 20.0 (5) Sodium Lauryl Sulfoacetate 5.0 (6) L-Glutathione 0.01 (7) Zinc Salicylate 1.11 (8) Protodioscin 0.1 (9) Tigogenin 0.11 (10) Ascorbic acid 10.0 (11) Phenoxyethanol 0.7 (12) Ethylhexylglycerin 0.3 (13) Ethyl lactate 5.0 (14) Fragrance 0.2. Procedure. Mix (1) and (2) to a clear thin gel. Add all other ingredients and mix in a homogenizer. A white cream-like cleanser is obtained.

### Example 8

Antiaging Gel with Facial Oil Reduction.

Ingredients. (1) Triethyl Citrate 67.00 (2) Ethylenediamine/Hydrogenated Dimer Dilinoleate Copolymer Bis-Di-C14-18 Alkyl Amide 10.0 (3) Ximenia Oil 0.1 (4) Sesamin 1.0 (5) Magnolol (and Honokiol 0.2 (6) Schisandrin 0.5 (7) Tetrahydrocurcuminoids 0.2 (8) Zeolite 20.0 (9) Fragrance 1.0. Procedure. Mix (1) and (2) and heat at 80 to 90 C till clear. Cool to 40 to 50 C and add all other ingredients and mix. Cool to room temperature. A translucent gel-like product is obtained.

### Example 9

Wound Healing Transparent Gel.

Ingredients. (1) C12-15 Alkyl Benzoate 92.00 (2) Dibutyl Lauroyl Glutamide 1.0 (3) Ximenia Oil 0.1 (4) Triethyl citrate 5.0 (5) Magnolol (and Honokiol 0.2 (6) Paeonol 0.5 (7) Tetrahydrocurcuminoids 0.2 (8) Silybin 1.0. Procedure. Mix (1) and (2) and heat at 95 to 110 C till clear. Cool to 40 to 50 C and add all other ingredients and mix. Cool to room temperature. A transparent gel-like product is obtained.

### Example 10

Dandruff Control Spray Lotion.

Ingredients. (1) PEG-4 81.0 (2) Triethyl Citrate 16.0 (32) Fragrance 0.5 (4) Paeonol 0.5 (5) Sesamin 2.0. Procedure. Mix all ingredients till a clear solution is obtained. Fill in spray bottles.

### Example 11

Dandruff Reduction Shampoo.

Ingredient. (1) Water 60.7 (2) Verbascoside 1.2 (3) Sodium Lauryl Sulfoacetate 10.0 (4) Disodium Laureth Sulfosuccinate 20.0 (5) Phenoxyethanol 0.7 (6) Chlorphenesin 0.3 (7) PEG-120 Methyl Glucose Dioleate 2.5. (8) Zinc salicylate 1.5 (9) Ethyl lactate 2.5 (10) Oat Extract 0.1. (11) Ethylhexylglycerin 0.5. Procedure. Mix (1) to (7) and heat at 60 to 70 C to a clear solution. Cool to 35 to 40 C and add all other ingredients and mix. Cool to room temperature.

### Example 12

Diaper Rash Reduction Lotion.

Ingredients. (1) Water 69.36 (2) Acrylates/C10-30 Alkyl Acrylate Crosspolymer 0.5 (3) Escin 0.1 (4) Sodium Stearyl Phthalamate 1.0 (5) Sodium Hydroxide 0.14 (6) Cetyl Alcohol 4.0 (7) Phenoxyethanol 0.7 (82) Chlorphenesin 0.3 (9) Triethyl Citrate 5.0 (10) Ethylhexylglycein 0.5 (11) Silymarin 2.0 (12) PEG-6 10.0 (13) Tetrahydrocurcuminoids 0.1 (14) Magnolol 0.1 (15) Paeonol 0.2 (16) Ethyl lactate 5.0 (17) Fragrance 1.0. Procedure. Mix (1) to (11) and heat at 80 to 90 C till clear. Cool to 45 to 55. Pre-mix (12) to (16) and add to main batch and mix. Cool to room temperature and adjust pH to 7.5.

### Example 13

Sunburn Treatment Lotion.

Ingredients. (1) Water 67.86 (2) Acrylates/C10-30 Alkyl Acrylate Crosspolymer 0.5 (3) Carnosine 0.1 (4) Sodium Stearyl Phthalamate 1.0 (5) Sodium Hydroxide 0.14 (6) Cetyl Alcohol 4.0 (7) Phenoxyethanol 0.7 (8) Sesamin 0.3 (9) Triethyl Citrate 10.0 (10) Silybin 1.0 (11) Ethylhexylglycein 0.5 (12) Polysorbate-20 2.0 (13) PEG-6 10.0 (14) Tetrahydrocurcuminoids 0.1 (15) Magnolol 0.1 (16) Copper Adenosine Triphosphate 0.2 (17) Paeonol 0.5 (18) Fragrance 1.0. Procedure. Mix (1) to (17) and heat at 80 to 90 C till clear. Cool to 35 to 45. Add (18) and mix. Cool to room temperature and adjust pH to 7.5. A lotion is obtained.

### Example 14

The Two-Step Skin Discomfort Treatment Method Using a Lignan in a Burn Treatment Gel Base.

The preparation of a lignan penetration enhancing composition by combination of a lignan, having general chemical structure according to FIG. 1, with a solubilizing-penetration enhancing agent is first performed by using the following ingredients and following the mixing and application procedures noted below.
1. Verbascoside 5.0
2. PEG-6 46.5
3. NH4 Acryloylmethyltaurate 1.0
4. Diglycerol 4.0
5. Silicone Wax 6.0
6. Deionized Water 20.0
7. Glycerin 5.0
8. Preservative 0.5
9. Vitamin E 2.0
10. Dimethicone 4.0
11. Dimethiconol 4.0
12. Cetyl Dimethiconol 2.0

Mixing Procedure: The ingredients 3 and 6 were mixed and heated at 40 to 50 C for 30 minutes. All other ingredients were then added to it with mixing. The composition was cooled to room temperature. A translucent gel was obtained. Application Procedure: The above penetration composition was applied to the afflicted area of skin where topical benefits are desired, and, wherein said solubilizing-penetration enhancing agent causes the penetration of said lignan into the dermis layer of skin, and steps (i) and (ii) repeated until the desired benefits are obtained.

### Example 15

The Two-Step Skin Discomfort Treatment Method using a High Potency Anti-Aging and Skin Brightening Composition.

The preparation of a lignan penetration enhancing composition by combination of a lignan, having general chemical structure according to FIG. 1, with a solubilizing-penetration enhancing agent is first performed by using the following ingredients and following the mixing and application procedures noted below.
1. Rosmarinic Acid 9.0
2. PEG-6 25.0
3. Diglycerol 10.0
4. Deionized Water 50.0
5. Silicone Emulsion 5.0
6. Preservative 1.0

Mixing Procedure: All ingredients were mixed at 50 to 60 C. A composition of serum consistency was obtained. Application Procedure: The above penetration composition was applied to the afflicted area of skin where topical benefits are desired, and, wherein said solubilizing-penetration enhancing agent causes the penetration of said lignan into the dermis layer of skin, and steps (i) and (ii) repeated until the desired benefits are obtained.

### Example 16

The Two-Step Skin Discomfort Treatment Method Using a Lignan in a Cosmetic Gel Base for Acne.

The preparation of a lignan penetration enhancing composition by combination of a lignan, having general chemical structure according to FIG. 1, with a solubilizing-penetration enhancing agent is first performed by using the following ingredients and following the mixing and application procedures noted below.
1. PEG-6 57.60
2. Aristoflex AVC 1.0
3. Glycerin 5.0
4. Water 20.0
5. Preservative 0.5
6. Vitamin E 0.5
7. Zn Salicylate 2.0
8. Silybin 2.0
9. Dimethicone 4.0
10. Dimethiconol 4.0
11. Cetyl Dimethiconol 2.0
12. Galanga Ext. 0.2
13. Esculin 0.5
14. Boswellia Serrata Ext. 0.2
15. Methylsulfonylmethane 0.5

Procedure: Mix all ingredients and heat at 60 to 70 C for 30 minutes. Cool to room temperature. A clear pale yellow gel of composition was obtained. Application Procedure: The above penetration composition was applied to the afflicted area of skin where topical benefits are desired, and, wherein said solubilizing-penetration enhancing agent causes the penetration of said lignan into the dermis layer of skin, and steps (i) and (ii) repeated until the desired benefits are obtained.

### Example 17

A Method for Topical Inhibition of Fatty Acid Desaturase using Lignans in a Cosmetic Gel Base for Rash Treatment.

The Method for Topical Inhibition of Fatty Acid Desaturase is preceded by the preparation of a lignan penetration enhancing composition by combination of a lignan, having general chemical structure according to FIG. 1, with a solubilizing-penetration enhancing agent is first performed by using the following ingredients and following the mixing and application procedures noted below.
1. Glycerin 54.16
2. PEG-6 7.0
3. Water 20.0
4. Preservative 0.5
5. Dow Corning 2501 10.0
6. Structure Plus 4.0
7. Eyebright Ext. 0.1
8. Botanical Ext. 0.1
9. Vitamin E 0.1
10. Sesamin 0.9
11. Schisandrin 1.76
12. Verbascoside 1.38

Procedure: All the ingredients were mixed and heated at 60 to 70 C for 30 minutes. The product is obtained as a light yellow gel, useful for face and neck zone anti-acne, anti-age and anti-wrinkle applications. The product has the pH of 4.5. Application Procedure: The above penetration composition was applied to the afflicted area of skin where topical benefits are desired, and, wherein said solubilizing-penetration enhancing agent causes the penetration of said lignan into the dermis layer of skin and inhibition of Fatty Acid Desaturase. The steps (i) and (ii) repeated until optimal desired benefits are obtained.

### BRIEF DESCRIPTION OF THE DRAWINGS

[FIG. 1] Lignan Structures

[FIG. 2] Examples of Lignans and Analogs.

[FIG. 3] Inhibition of Desaturase by Silybin.

Inhibition of Desaturase by Silybin.

1. A two-step skin discomfort treatment method comprising; (i) the preparation of a lignan penetration enhancing composition by combination of a lignan, having general chemical structure according to FIG. 1, with a solubilizing-penetration enhancing agent, and (ii) the application of said penetration enhancing composition to the afflicted area of skin where topical benefits are desired, and, wherein said solubilizing-penetration enhancing agent causes the penetration of said lignan into the dermis layer of skin, and (iii) repeating of steps (i) and (ii) until the desired benefits are obtained.

2. A treatment method according to claim 1, wherein said lignan is selected from silybin, silymarin, silydianin, silychristin isosilybin, sauriol, licarin, saucernetin, saucerneol, niranthin, Phyllanthin, manassantins, matairesinol, hydroxymatairesinol, oxomatairesinol, saminol, americanin, arctiin, arctigenin, lariciresinol, isolariciresinol, secoisolariciresinol, secoisolariciresinol diglycoside, rubrisandrin, egonol, masutakeside, styraxlignolide, lappaol, diarctigenin, interiotherin, schisandrol, schisandrin, sesamin, sesaminol, episesamin, episesaminol, sesamolin, verbascoside, tetrahydrocurcumin, rosmarinic acid, chlorogenic acid, guaiaretic acid, dihydroguiaretic acid, nor-dihydroguiaretic acid, alpha-conidendrin, liovil, picearesinol, syringaresinol, nortrachelogenin; their analogs and derivatives, or combinations thereof.

3. A treatment method according to claim 1, wherein said lignan is derived from a plant and/or extract of such plant.

4. A treatment method according to claim 1, wherein said lignan is Sesamin.

5. A treatment method according to claim 1, wherein said solubilizing-penetration enhancing agent is selected from the group comprising hydroxy acid esters, which includes methyl lactate, ethyl lactate, propyl lactate, isopropyl lactate, butyl lactate, isobutyl lactate, t-butyl lactate, pentyl lactate, neopentyl lactate, isopentyl lactate, hexyl lactate, ethylhexyl lactate, glycerol lactate, benzyl lactate, triethyl citrate, trimethyl citrate, tributyl citrate, acetyl triethyl citrate, acetyl tributyl citrate, trihexyl citrate, butyl trihexyl citrate, stearyl citrate, diethyl tartrate, dimethyl tartrate, ethyl mandelate, ethyl salicylate, methyl salicylate, ethyl glycolate, and combinations thereof.

6. A treatment method according to claim 1, wherein said solubilizing-penetration enhancing agent is selected from the group comprising glycols, which includes glycol ethers, glycol ether esters, glycol ether amides, poly ethylene glycol, polypropylene glycol, polyglycerin, diglycerin, ethoxydiglycol, methylpropanediol, and combinations thereof.

7. A treatment method according to claim 1, wherein said solubilizing-penetration enhancing agent is Triethyl citrate.

8. A treatment method according to claim 1, wherein said solubilizing-penetration enhancing agent is ethoxydiglycol.

9. A treatment method according to claim 1, wherein said skin discomfort is from acne.

10. A treatment method according to claim 1, wherein said skin discomfort is from wounded skin including abrasions, bites, and surgical wounds.

11. A treatment method according to claim 1, wherein said skin discomfort is from dandruff including dandruff-related hair loss.

12. A treatment method according to claim 1, wherein said skin discomfort is from a burn including thermal burn, radiation burn, razor burn, and sunburn.

13. A treatment method according to claim 1, wherein said skin discomfort is from sting, including diaper rash sting, insect sting, and shaving sting.

14. A treatment method according to claim 1, wherein a cosmetically or pharmaceutically acceptable carrier base is included.

15. A treatment method according to claim 1, wherein carrier base is a semi-solid composition including a gel or cream.

16. A treatment method according to claim 14, wherein carrier base further includes skin cleansers, surfactants, skin conditioning agents, hair conditioning agents, vitamins, hormones, minerals, plant extracts, anti-inflammatory agents, emollients, moisturizers, humectants, silicones, skin soothing ingredients, analgesics, skin penetration enhancers, solubilizers, anesthetics, colorants, perfumes, preservatives, seeds, broken seed nut shells, silica, clays, beads, luffa, polyethylene balls, mica, pH adjusters, processing aids, and combinations thereof.

17. A method for topical inhibition of Fatty Acid Desaturase, comprising (i) the mixing of a lignan with a carrier agent, and (ii) the application of said mixture on skin in an effective amount, and (iii) wherein said carrier causes the penetration of said lignan into the dermis layer of skin, and said lignan causes the inhibition of said Fatty Acid Desaturase therein.

18. A method according to claim 17, wherein said method is for the reduction of skin wrinkles.

19. A method according to claim 17, wherein said method is for reduction of skin pigmentation.

20. A method according to claim 17, wherein said method is for the reduction of dandruff and dandruff-related hair loss.

21. A method according to claim 17, wherein said method is for the control of oily skin including acne caused by oily skin.

22. A method according to claim 17, wherein said method is for the treatment of rash and rash caused by sting.

23. A method according to claim 17, wherein said carrier agent is a semi-solid composition including a gel or cream.

24. A composition comprising a lignan, having general chemical structure according to FIG. 1, for treatment of topical discomforts and ailments selected from the group comprising sunburn, radiation burn, thermal burn and blisters, diaper rash, shaving burn, wound, venous cannulation, acne, darkened skin pigmentation, skin wrinkles, dandruff, and hair loss.
